(19) United States
(12) Patent Application Publication
(10) Pub. No.: US 2015/0056655 A1
(43) Pub. Date: Feb. 26, 2015

(54) EXPRESSION CONSTRUCTS AND METHODS FOR EXPRESSING POLYPEPTIDES IN EUKARYOTIC CELLS

(71) Applicant: Glenmark Pharmaceuticals S.A., La Chaux-de-Fonds (CH)

(72) Inventors: Christel AEBISCHER-GUMY, La Chaux-de-Fonds (CH); Martin Bertschinger, La Chaux-de-Fonds (CH); Pierre Moretti, La Chaux-de-Fonds (CH)

(21) Appl. No.: 14/453,328

(22) Filed: Aug. 6, 2014

(30) Foreign Application Priority Data
Aug. 6, 2013 (EP) 13179375.4

(51) Int. Cl. C07K 16/46; C12N 15/85

(52) U.S. Cl. CPC C07K 16/468; C12N 15/85; C07K 2317/31; C12N 2840/445; C12N 2840/20
USPC 435/69.6; 435/320.1; 435/328

(57) ABSTRACT

The invention relates to an expression construct for the expression of polypeptides in host cells using alternative splicing. The expression construct can be used for the expression of polypeptides such as antibodies, antibody fragments and bispecific antibodies by expressing the gene products required for protein expression at the ratio leading to the highest titres or the best product quality profile.

### CROSS-REFERENCE TO RELATED APPLICATIONS

This application claims the benefit of European Patent Application No. 13179375.4, filed Aug. 6, 2013, which is incorporated by reference herein in its entirety.

### REFERENCE TO A SEQUENCE LISTING SUBMITTED ELECTRONICALLY VIA EFS-WEB

The content of the electronically submitted sequence listing (Name: 3305_{—}0160000_SEQLISTING.ascii.txt; Size: 290,260 bytes; and Date of Creation: Aug. 5, 2014) is herein incorporated by reference in its entirety.

### BACKGROUND OF THE INVENTION

1. Field of the Invention

The present invention relates to expression constructs and methods for expressing polypeptides and/or polypeptide multimers in eukaryotic cells using alternative splicing. Methods for producing host cells containing these constructs are included, as well as the use of these constructs and the polypeptides expressed therefrom for the efficient production of proteins.

2. Background Art

In order to produce a protein in a eukaryotic cell, the DNA coding for this protein has to be transcribed into a messenger RNA (mRNA) which will in turn be translated into a protein. The mRNA is first transcribed in the nucleus as pre-mRNA, containing introns and exons. During the maturation of the pre-mRNA into mature mRNA, the introns are cut out (“spliced”) by a protein machinery called the spliceosome. The exons are fused together and the mRNA is modified by the addition of a so called CAP at its 5′ end and a poly(A) tail at its 3′ end. The mature mRNA is exported to the cytoplasm and serves as template for the translation of proteins which are encoded therein.

Alternate splicing is a term describing the phenomenon wherein the same pre-mRNA transcript might be spliced in different fashions leading to different mature mRNAs and in some cases to different proteins. This mechanism is used in nature to change the expression level of proteins or in order to modify the activity of certain proteins during development (Cooper T A & Ordahl C P (1985), J Biol Chem, 260(20): 11140-8). Alternate splicing is usually controlled by complex interactions of many factors (Orengo J P et al., (2006) Nucleic Acids Res, 34(22): e148).

Although splicing is well known in the literature and consensus sequences have been published for splicing in human cells, the precise outcome of alternate splice events is not easy to predict due to multiple factors that might influence the splicing. Factors known to influence splicing include the consensus sequences of the branch point, the splice donor and the splice acceptor region, the size of the exon and the intron, and binding sites for regulatory proteins leading to increased or reduced splicing (see Alberts B et al (2002) Molecular Biology of the Cell, 4th edition, New York: Garland Science).

Alternate splicing can be used in order to increase the expression level of polypeptides, particularly, multimeric proteins, for example antibodies. The level of antibody expression depends on the ratio of heavy chain to light chain expression. Although the literature suggests that it is favourable to express more light chain than heavy chain (Dorai H et al., (2006) Hybridoma (Larchmt), 25(1): 1-9), the applicants have determined that the optimal ratio of light to heavy chain leading to maximum expression is largely dependent on the antibody. The same is true for bispecific antibodies, where the inventors have shown that the antibody expression level depends on the ratio of the different chains that form the bispecific antibody.

Methods for expressing polypeptides in host cells using alternative splicing have been described previously in the art. For example, Prentice (WO200589285) describes an expression vector that comprises two or more expression cassettes under the control of a single promoter where the expression cassettes have splice sites which allow for their alternative splicing. In this construct, a polyadenylation (poly(A)) site is included after each open reading frame. Similarly, Fallot et al (WO2007135515) also describe an expression cassette that can be expressed in a host cell using a single promoter to drive transcription of a pre-mRNA which can be spliced into two or more mRNAs for subsequent polypeptide expression. This expression cassette comprises a polyadenylation signal located at its 3′ end, which, according to the applicants, avoids any additional regulation involving competition between the splice sites and transcription termination processes. In addition, an IRES operably linked to a selection marker is also included before the 3′ polyadenylation signal in order to enable selection of stable cell lines. An alternative construct from Lucas et al., (Nucleic Acids Research, 1996, 24(9): 1774-9) comprises only one intron, one splice donor and one splice acceptor site, where the intron is either spliced or not.

Alternate splicing could be used in order to express the subunits needed for an antibody at the ratio leading to the highest titers. For example a heavy chain and a light chain are cloned on the same construct. Splicing will lead to a specific ratio of mRNA expressing the heavy chain or the light chain. This ratio could be adjusted to be close to the optimum for the expression of the final antibody. In the production of bispecific molecules the ratio might affect not only the expression levels, but also the product quality. The optimal ratio could be identified by looking at the highest expression of the product species of interest. It could also be beneficial to choose a ratio with minimal by-product production.

### SUMMARY OF THE INVENTION

The present invention relates generally to expression systems such as expression constructs and expression vectors which can be used to obtain increased expression and to optimize product quality in recombinant polypeptide production. Using an expression construct as described herein, high transient and stable titers can be obtained, which for transient expression were found to be up to 60 times higher compared to transient titres observed in previous, prior art studies.

In a first aspect, the present invention relates to an expression construct that can be used for the efficient expression of polypeptides. Preferably, the expression construct comprises in a 5′ to 3′ direction:

a promoter,

an optional first splice donor site;

a first flanking intron;

a splice acceptor site;

a first exon encoding a first polypeptide;

an optional second splice donor site;

a second flanking intron;

a splice acceptor site; and

a second exon encoding a second polypeptide,

wherein upon entry into a host cell, transcription of the first exon results in expression of the first polypeptide and/or transcription of the second exon results in expression of the second polypeptide.

The inventors of the present invention have found that use of flanking introns or fragments thereof before and after the first exon and which share at least 80% nucleic acid sequence homology with each other, has a significant impact on the level of polypeptide expression. In an embodiment of the present invention, the introns flanking the first exon can be derived from naturally occurring introns that are alternately spliced, and also from constitutively spliced introns. Preferably, the introns can be selected from the group consisting of: chicken troponin (cTNT) intron 4, cTNT intron 5 and introns of the human EF1alpha gene, preferably the first intron of the human EF1alpha gene. More preferably, the introns flanking the first exon are derived from chicken troponin intron 4 (cTNT-I4). Preferably, the flanking introns share 80% nucleic acid sequence homology, more preferably 90% nucleic acid sequence homology and most preferably 95% nucleic acid sequence homology. In a further preferred embodiment of the present invention, the flanking introns share 98% nucleic acid sequence homology. In a most preferred embodiment of the present invention, the flanking introns share 100% nucleic acid sequence homology and have an identical nucleic acid sequence. The percentage of sequence homology between the flanking intron sequences may be determined by comparing a stretch of nucleic acids excluding the poly(Y) tract sequence.

Preferably, the flanking introns share homology for a stretch of nucleic acid of at least 50 nucleotides in length. Preferably the flanking introns share homology along a stretch of nucleic acid of at least 50 to 100 nucleotides in length, preferably of at least 50 to 150 nucleotides in length, preferably of at least 50 to 200 nucleotides in length, preferably of at least 50 to 250 nucleotides in length, more preferably of at least 50 to 300 nucleotides in length, more preferably of at least 50 to 350 nucleotides in length, even more preferably of at least 50 to 400 nucleotides in length and most preferably of at least 50 to 450 nucleotides in length. In an embodiment of the present invention, the maximum length of the flanking intron is 450 nucleotides.

In an aspect of the present invention, the expression construct comprises at least one polypyrimidine (poly(Y)) tract. This can be located between the branch point and the splice acceptor, upstream of the first exon. In one embodiment, reducing the number of pyrimidine bases in the poly(Y) tract leads to an increase in expression of the second polypeptide from the second exon. The number of pyrimidine bases present in the poly(Y) tract can be 30 or less, preferably 20 or less, more preferably 10 or less, even more preferably 7 or less and most preferred 5 or less. Alternatively the poly(Y) tract can be located downstream of the first exon.

In a further aspect of the present invention, the second splice donor site is eliminated. In a preferred embodiment, the elimination of the second splice donor site is combined with a reduction in the number of pyrimidine bases in the poly(Y) tract upstream of the first exon.

In another embodiment of the present invention, the expression construct further comprises a 5′UTR, a third splice donor site, an intron, a third splice acceptor site and a further 5′UTR. Preferably, the splice donor site, intron and splice acceptor site are constitutive such that the intron is constitutively spliced in the mature mRNA. Preferably these constitutive components are located between the promoter and the splice donor site preceding the first flanking intron.

In a preferred embodiment of the present invention a polyadenylation (poly(A)) site is not present within the expression construct. Preferably a poly(A) site will be present at the end of the expression construct.

The flanking intron sequence starting from the branch point to the start of the following exon, generated in the present invention, are all unique artificial sequences. Preferably, these artificial sequences are comprised in the sequences selected from the group consisting of SEQ ID Nos: 38 to 128. More preferably, the artificial sequences have the sequence starting from the branch point to the start of the following exon and are selected from the group consisting of SEQ ID Nos: 129 to 175.

In an aspect of the present invention, the polypeptides encoded by the first and second exons can be protein multimers i.e. heteromultimeric polypeptides such as recombinant antibodies or fragments thereof. The antibody fragments may be selected from the list consisting of: Fab, Fd, Fv, dAb, F(ab′)₂ and scFv. In one embodiment, the first polypeptide expressed by the expression construct can be an antibody heavy chain or an antibody light chain or fragments thereof. Where the first polypeptide expressed is an antibody heavy chain, the second polypeptide expressed by the expression construct is an antibody light chain. Alternatively, where the first polypeptide expressed is an antibody light chain, the second polypeptide is an antibody heavy chain.

In a further aspect of the present invention, the expression construct can be used for the expression of a bispecific antibody in a host cell. In one embodiment, the first polypeptide expressed is an antibody heavy chain and the second polypeptide expressed is a fragment of antibody linked to an antibody Fc region. The antibody fragment may be selected from the list consisting of: Fab, Fd, Fv, dAb, F(ab′)₂ and scFv. Preferably the antibody fragment is a Fab or a scFv. More preferably the antibody fragment is a scFv.

In addition, a separate expression construct may be provided for the expression of an antibody light chain in a host cell. Co-expression of the expression construct coding for an antibody heavy chain and an antibody fragment-Fc with an expression construct coding for an antibody light chain in host cells, can result in the expression of a bispecific antibody. In a further preferred embodiment of the invention the Fc region of the antibody heavy chain and the Fc region linked to the antibody fragment expressed by the first and second polypeptides comprise a modification such that the interaction of these Fc regions is enhanced. Furthermore, the modification to the Fc regions may result in increased stability of the bispecific antibody.

### BRIEF DESCRIPTION OF THE FIGURES

FIG. 1a: Schematic drawing of an alternate splicing construct of the present invention. The construct contains four exons. The exon 1 and exon 2 are separated by the first intron (AS intron #1), which is constitutively cut out by the splice machinery of the cell. Exon 3 (referred to as “alternate exon”) is either included or cut out. It contains the first open reading frame coding for dsRED. This exon is flanked upstream by AS intron #2, which (in the basic construct) is derived from chicken troponin intron 4 (cTNT-I4) and downstream by AS intron #3 which is (in the basic construct) derived from chicken troponin intron 5 (cTNT-I5). Exon 4 is constitutively included in the mRNA. Nevertheless the open reading frame coding for GFP is only expressed if it is the first open reading frame on the mature mRNA. Therefore, if the alternate exon 3 is included in the construct, only dsRED encoded on exon 3 will be translated (on top of the drawing). If exon 3 was spliced out, exon 4 contains the first open reading frame of the mRNA and GFP will be expressed (on the bottom of the drawing).

FIG. 1b: Example of gating applied for FACS results analysis: only transfected cells were considered and separated into four populations: dsRED⁻GFP⁺, dsRED⁺GFP⁺⁺, dsRED⁺⁺GFP⁺ and dsRED⁺GFP⁻. The percentage of transfected cells in each of these populations was considered for results analysis.

FIGS. 2a, b and c: Details of the splicing constructs. (2a) Modifications in the splice acceptor site of the alternate exon containing the open reading frame for dsRED. The modifications include the number of pyrimidines (Ys; the bases C and T) in the region between the branch point and the intron-exon consensus region that is called the poly(Y) tract, modifications in the branch point regions and modifications in the intron-exon consensus sequence. (2b) Modifications in the poly(Y) tract of the second splice acceptor upstream of the exon coding for GFP. In the original construct cTNT-I5 was used. The poly(Y) tract was enriched in Y. Compared to the original construct (I5), the amount of Ys were increased by a factor of almost 3. (2c) Elimination of the splice donor site of cTNT-I4 located downstream of the alternate exon. Shown is an alignment of the native I4 sequence and the shortened version I4(sh), that lacks the exon-intron consensus sequence.

FIGS. 3a and b: Transient transfection of HEK293 (3a) or CHO-S (3b) cells of alternate splicing constructs with modifications in the poly(Y) tract. Gating was performed as described in FIG. 1. The numbers represent the percentage of the respective population (dsRED⁻GFP⁺, dsRED⁺GFP⁺⁺, dsRED⁺⁺GFP⁺ and dsRED⁺GFP⁻) of transfected cells. The basal construct GSC2250 shows a strong preference for the expression of dsRED (on exon #3, the alternate exon—see FIG. 1) over GFP (on exon #4—see FIG. 1). The content of Ys in the poly(Y) tract of AS intron #2 was decreased in order to weaken the splice acceptor site of the exon coding for dsRED and the content of Ys in the poly(Y) tract of AS intron #3 was increased in order to strengthen the splice acceptor site of the exon coding for GFP. A significant, but modest shift was observed for decrease of the splice acceptor site of the exon coding for dsRED, especially for constructs 5Y-5, 5Ynude and 0Y. No effect could be observed for the increase of the splice acceptor site of the exon coding for GFP. The general trend was the same for CHO-S and HEK293 cells. As a positive control, cells were transfected only with GFP or with dsRED.

FIGS. 4a and b: Modification in the branch point region and the intron-exon consensus sequence (top row of 4a and 4b, respectively) and of the intron arrangements (middle row of 4a and 4b, respectively) for HEK293 cells (4a) and CHO-S cells (4b). Bottom row of (4a) and (4b), respectively: As a positive control cells were transfected with dsRED or GFP only. The construct GSC2250 was included as reference for the splice ratio of the basal construct (cTNT-I4|cTNT-I5). The numbers represent the percentage of the respective population (dsRED⁻GFP⁺, dsRED⁺GFP⁺⁺, dsRED⁺⁺GFP⁺ and dsRED⁺GFP⁻) of transfected cells. Gating was performed as described in FIG. 1.

FIGS. 5a and b: Sequence modification of the branch point region and reduction of Ys in the poly(Y) tract of construct cTNT-I4|cTNT-I4. (5a) Transfection of HEK293 cells. Top row: The reduction of the amount of Ys in the poly(Y) tract has a major impact on the expression of GFP. Middle row: Modifications in the branch point region. No major increase in expression of GFP could be identified. Bottom row: Cells were transfected with dsRED or GFP only. The construct GSC2250 was included as reference for the splice ratio of the basal construct. (5b) Transfection of CHO-S cells. Setup of experiment was equivalent to top and bottom rows of (5a) and results are similar. The numbers represent the percentage of the respective population (dsRED⁻GFP⁺, dsRED⁺GFP⁺⁺, dsRED⁺⁺GFP⁺ and dsRED⁺GFP⁻) of transfected cells. Gating was performed as described in FIG. 1.

FIG. 6: Elimination of the second splice donor site further shifts the alternative splicing ratio. The transfection was done in CHO-S cells. In some constructs, the elimination of the second splice donor site was combined with the reduction of the poly(Y) tract in the flanking region of the first exon. Here the shift of the alternative splicing towards the second open reading frame was even more pronounced. dsRED and GFP were transfected in the respective cells and used as controls. The basic construct cTNT-I4|cTNT-I4 was included in order to serve as control for the splice ratio of previous constructs. The numbers represent the percentage of the respective population (dsRED⁻GFP⁺, dsRED⁺GFP⁺⁺, dsRED⁺⁺GFP⁺ and dsRED⁺GFP⁻) of transfected cells. Gating was performed as described in FIG. 1.

FIG. 7: Schematic drawing of dsRED expression versus GFP expression. The alternate splicing event has a different equilibrium depending on the construct. Constructs were made that either expressed a majority of dsRED, intermediate amounts of dsRED and GFP, or a majority of GFP.

FIG. 8: Exemplary GFP and dsRED expression of eight randomly chosen clones.

FIG. 9: Sequence alignment of constructs.

FIG. 10: Expression results of constructs expressing an anti-HER2 antibody in the pGLEX3 backbone. The constructs are ordered first by order of the alternate exon and second by decreasing order of poly(Y) in the construct. The two constructs expressing best are for the orientation LC-HC: I4(0Y)-I4 and for the orientation HC-LC: I4(7Ynude)-I4sh.

FIG. 11: Fine tuning of an anti-HER2 antibody alternate splicing cassette using intron-exon consensus region modifications and branch point mutations. After preselection of constructs listed in Table 7 in 12 well plate scale (data not shown), selected constructs were reassessed in tubespin scale. The titers have been determined on day 6 after transfection using the Octet device (Fortebio, Melo Park, Calif.).

FIG. 12: Identical introns upstream and downstream of the alternate exon lead to higher expression. For the two different orientations the highest expression was observed if the same intron was used before and after the alternate exon. Using the cTNT-I4 intron flanking the alternate exon, the expression level was shown to be highest.

FIG. 13: Expression level of 72 minipools in tubespin 50 ml bioreactor format at the end of a 2 week supplemented batch at 37° C., 5% CO2, and 80% humidity on a shaken bioreactor. The clones are ranked by decreasing expression level.

FIG. 14: Expression level of the best 23 clones for parental minipools #68, 164 and 184, and the best 25 clones for parental minipool #148 respectively, in tubespin 50 ml bioreactor format at the end of a 2 week supplemented batch at 37° C., 5% CO2, and 80% humidity on a shaken bioreactor. The expression level of the parental minipool is shown in open bars, the expression of the clones derived from the respective minipool in closed bars.

FIG. 15: Expression level of the alternate splicing construct co-transfected with the light chain at different ratios.

### DETAILED DESCRIPTION OF THE INVENTION

The present invention provides expression constructs and methods for expressing polypeptides, especially heteromultimeric polypeptides such as recombinant antibodies or fragments thereof or bispecific antibodies in host cells using alternative splicing. The invention provides a construct which may be expressed in a host cell using a single promoter to drive the transcription of a pre-mRNA which can be spliced into two or more mRNAs with the subsequent translation into different polypeptides.

The term “expression construct” or “construct” as used interchangeably herein includes a polynucleotide sequence encoding a polypeptide to be expressed and sequences controlling its expression such as a promoter and optionally an enhancer sequence, including any combination of cis-acting transcriptional control elements. The sequences controlling the expression of the gene, i.e. its transcription and the translation of the transcription product, are commonly referred to as regulatory unit. Most parts of the regulatory unit are located upstream of coding sequence of the gene and are operably linked thereto. The expression construct may also contain a downstream 3′ untranslated region comprising a polyadenylation site. The regulatory unit of the invention is either operably linked to the gene to be expressed, i.e. transcription unit, or is separated therefrom by intervening DNA such as for example by the 5′-untranslated region (5′UTR) of the heterologous gene. Preferably the expression construct is flanked by one or more suitable restriction sites in order to enable the insertion of the expression construct into a vector and/or its excision from a vector. Thus, the expression construct according to the present invention can be used for the construction of an expression vector, in particular a mammalian expression vector.

The term “polynucleotide sequence encoding a polypeptide” as used herein includes DNA coding for a gene, preferably a heterologous gene expressing the polypeptide.

The terms “heterologous coding sequence”. “heterologous gene sequence”, “heterologous gene”, “recombinant gene” or “gene” are used interchangeably. These terms refer to a DNA sequence that codes for a recombinant gene, in particular a recombinant heterologous protein product that is sought to be expressed in a host cell, preferably in a mammalian cell and harvested. The product of the gene can be a polypeptide. The heterologous gene sequence is naturally not present in the host cell and is derived from an organism of the same or a different species and may be genetically modified.

The terms “protein” and “polypeptide” are used interchangeably to include a series of amino acid residues connected to the other by peptide bonds between the alpha-amino and carboxy groups of adjacent residues.

The term “promoter” as used herein defines a regulatory DNA sequence generally located upstream of a gene that mediates the initiation of transcription by directing RNA polymerase to bind to DNA and initiating RNA synthesis. Promoters for use in the invention include, for example, viral, mammalian, insect and yeast promoters that provide for high levels of expression, e.g. the mammalian cytomegalovirus or CMV promoter, the SV40 promoter, or any promoter known in the art suitable for expression in eukaryotic cells.

The term “5′ untranslated region (5′UTR)” refers to an untranslated segment in the 5′ terminus of the pre-mRNA or mature mRNA. On mature mRNA, the 5′UTR typically harbours on its 5′ end a 7-methylguanosine cap and is involved in many processes such as splicing, polyadenylation, mRNA export towards the cytoplasm, identification of the 5′ end of the mRNA by the translational machinery and protection of the mRNAs against degradation.

The term “intron” refers to a segment of nucleic acid non-coding sequence that is transcribed and is present in the pre-mRNA but is excised by the splicing machinery based on the sequences of the donor splice site and acceptor splice site, respectively at the 5′ and 3′ ends of the intron, and therefore not present in the mature mRNA transcript. Typically introns have an internal site, called the branch point, located between 20 and 50 nucleotides upstream of the 3′ splice site. The length of the intron used in the present invention may be between 50 and 450 nucleotides long. A shortened intron may comprise 50 or more nucleotides. A full length intron may comprise up to 450 nucleotides.

The term “exon” refers to a segment of nucleic acid sequence that is transcribed into mRNA.

The term “splice site” refers to specific nucleic acid sequences that are capable of being recognized by the splicing machinery of a eukaryotic cell as suitable for being cut and/or ligated to a corresponding splice site. Splice sites allow for the excision of introns present in a pre-mRNA transcript. Typically the 5′ portion of the splice site is the referred to as the splice donor site and the 3′ corresponding splice site is referred to as the acceptor splice site. The term splice site includes, for example, naturally occurring splice sites, engineered splice sites, for example, synthetic splice sites, canonical or consensus splice sites, and/or non-canonical splice sites, for example, cryptic splice sites.

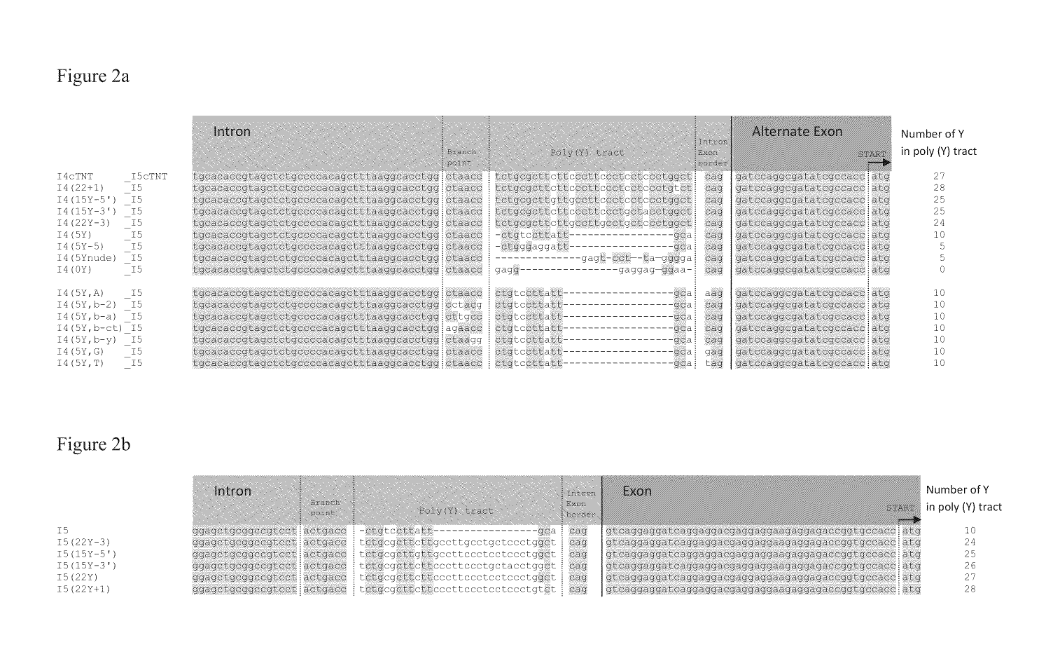

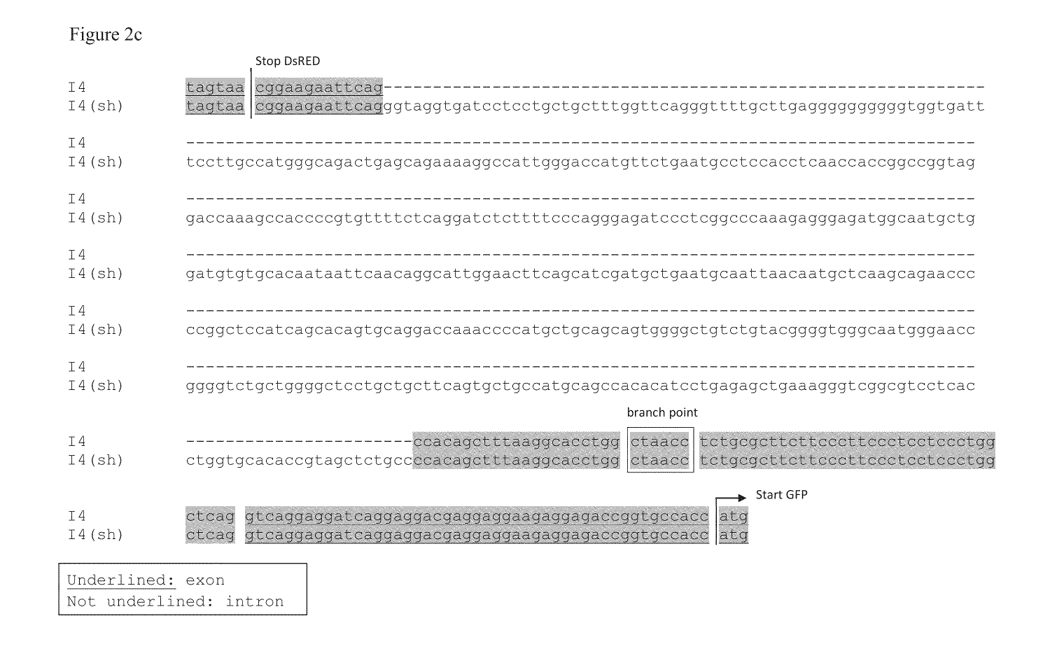

The term “poly(Y) tract” refers to the stretch of nucleic acids found between the branch point and the intron-exon border (illustrated in FIG. 2a or 2b). This stretch of nucleic acids has an abundance of polypyrimidines (Ys), meaning an abundance of the pyrimidine bases C or T.

The term “3′ untranslated region (3′UTR)” refers to an untranslated segment in the 3′ terminus of the pre-mRNAs or mature mRNAs. On mature mRNAs this region harbours the poly(A) tail and is known to have many roles in mRNA stability, translation initiation and mRNA export.

The term “enhancer” as used herein defines a nucleotide sequence that acts to potentiate the transcription of genes independent of the identity of the gene, the position of the sequence in relation to the gene, or the orientation of the sequence. The vectors of the present invention optionally include enhancers.

The term “polyadenylation signal” refers to a nucleic acid sequence present in the mRNA transcripts, that allows for the transcripts, when in the presence of the poly(A) polymerase, to be polyadenylated on the polyadenylation site located 10 to 30 bases downstream the poly(A) signal. Many polyadenylation signals are known in the art and may be useful in the present invention. Examples include the human variant growth hormone polyadenylation signal, the SV40 late polyadenylation signal and the bovine growth hormone polyadenylation signal.

The terms “functionally linked” and “operably linked” are used interchangeably and refer to a functional relationship between two or more DNA segments, in particular gene sequences to be expressed and those sequences controlling their expression. For example, a promoter and/or enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Orientation” refers to the order of nucleotides in a given DNA sequence. For example, an orientation of a DNA sequence in opposite direction in relation to another DNA sequence is one in which the 5′ to 3′ order of the sequence in relation to another sequence is reversed when compared to a point of reference in the DNA from which the sequence was obtained. Such reference points can include the direction of transcription of other specified DNA sequences in the source DNA and/or the origin of replication of replicable vectors containing the sequence.

The term “nucleic acid sequence homology” or “nucleotide sequence homology” as used herein include the percentage of nucleotides in the candidate sequence that are identical with the nucleotide sequence of the comparison sequence e.g. percentage of nucleotides in the first flanking intron that are identical with the nucleotide sequence of the second flanking intron, after aligning the sequences and introducing gaps, if necessary, to achieve the maximum percent sequence identity. Thus sequence identity can be determined by standard methods that are commonly used to compare the similarity in position of the nucleotides of two nucleotide sequences. Usually the nucleic acid sequence homology of the flanking intron sequences to each other is at least 80%, preferably at least 85%, more preferably at least 90%, and most preferably at least 95%, in particular 96%, more particular 97%, even more particular 98%, most particular 99%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%.

The term “expression vector” as used herein includes an isolated and purified DNA molecule which upon transfection into an appropriate host cell provides for a high-level expression of a recombinant gene product within the host cell. In addition to the DNA sequence coding for the recombinant or gene product the expression vector comprises regulatory DNA sequences that are required for an efficient transcription of the DNA coding sequence into mRNA and for an efficient translation of the mRNAs into proteins in the host cell line.

The term ‘about’ as used herein in relation to the length of a nucleic acid sequence, includes deviations of a maximum of ±50%, preferably of a maximum of ±10% of the stated values e.g. about 50 nucleotides includes values of 25 to 75 nucleotides, preferably 45 to 55 nucleotides, about 450 nucleotides includes values of 225 to 675 nucleotides, preferably 405 to 495 nucleotides.

The terms “host cell” or “host cell line” as used herein include any cells, in particular mammalian cells, which are capable of growing in culture and expressing a desired recombinant product protein.

Recombinant polypeptides and proteins can be produced in various expression systems such as prokaryotic (e.g. E. coli), eukaryotic (e.g. yeast, insect, vertebrate, mammalian), and in vitro expression systems. Most commonly used methods for the large-scale production of protein-based biologics rely on the introduction of genetic material into host cells by transfection of DNA vectors. Transient expression of polypeptides can be achieved with transient transfection of host cells. Integration of vector DNA into the host cell genome results in a cell line that is stably transfected and propagation of such a stable cell line can be used for the large-scale production of polypeptides and proteins.

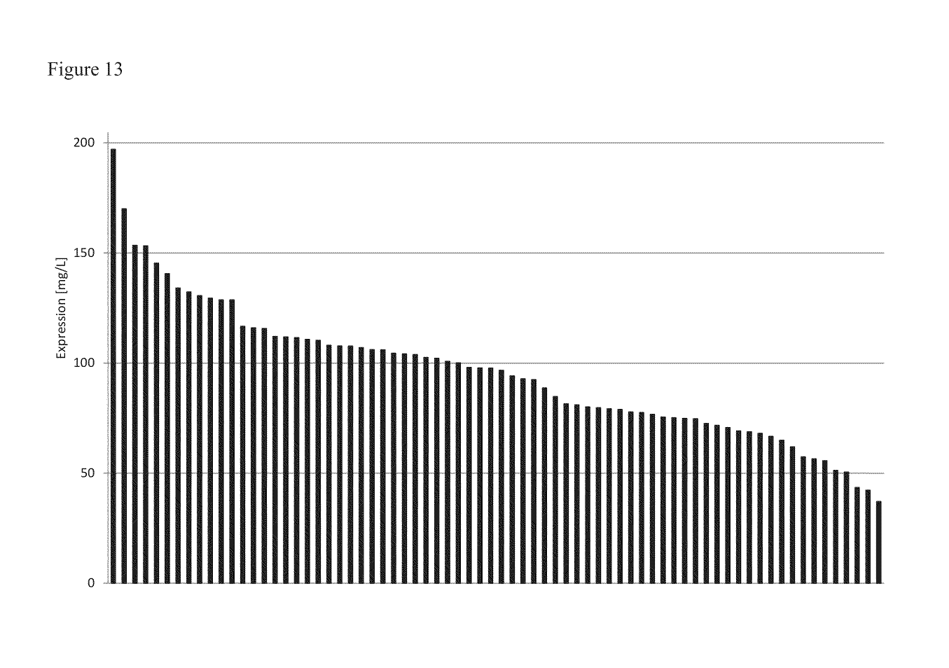

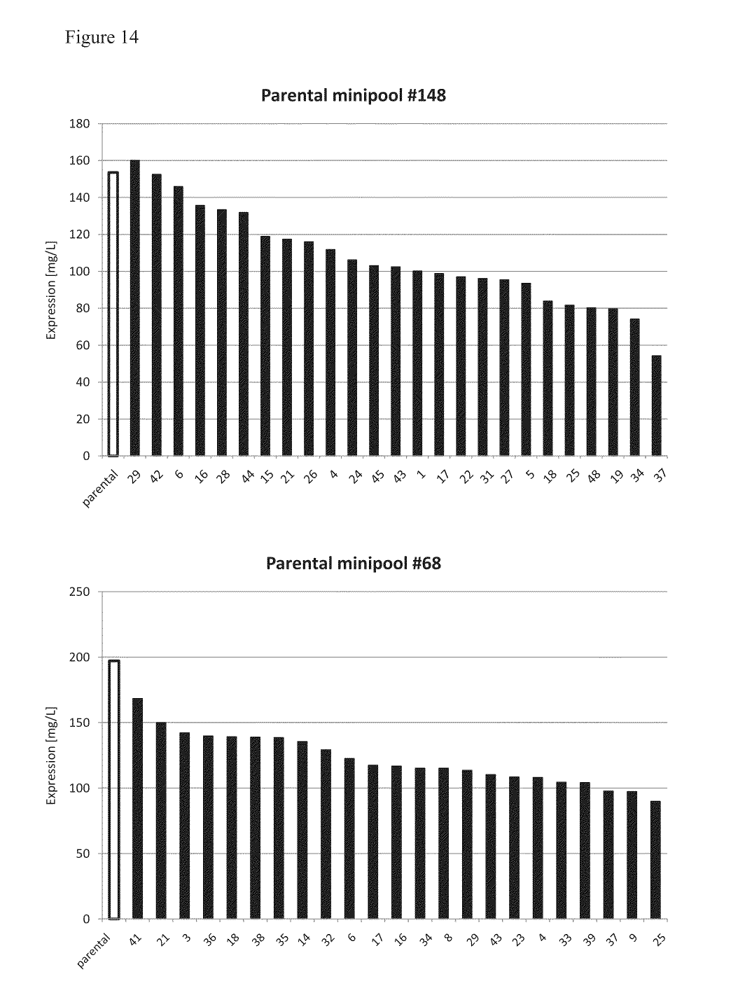

In contrast to the alternative splicing approaches described previously, the present applicants have designed an alternative splicing approach for the expression of polypeptides at a desired ratio through the use of multiple splice donor and acceptor sites in an expression construct. Such an approach enables high transient and stable titres of polypeptides to be produced, with transient titres of up to 60 times higher compared to those obtained in prior art approaches. For example, titres of up to 15 μg/ml of antibody were observed following transient transfection using an expression construct of the present invention, compared to levels of, for example, 0.25 μg/ml observed in Table 1 of WO200589285, supra. For stably transfected cell lines, titres of up to 200 μg/ml of antibody were observed in batch culture (FIG. 13), which was increased up to 250 μg/ml following a second round of limiting dilution (Example 4). In comparison to WO200589285, supra, where the highest titre of specific productivity of stable pools was observed to be 377 ng/ml (see Table 4 of WO200589285, supra), the titre level obtained by the present applicants was over 650 times higher, a vast increase over that observed in the prior art.

An expression construct of the present invention, comprises two alternate exons, each encoding a polypeptide. A splice donor site is included both upstream and downstream of the first exon. In addition, a splice acceptor site is included both upstream and downstream of the first exon. In a preferred embodiment of the present invention, the first exon is flanked by two functional copies of the same intron. During a splice event, these same intron sequences are cut out and are not present in the mature mRNA. Such a construct is functionally similar to naturally occurring alternate exons. Introns suitable for use in an expression construct of the present invention can be selected from the list consisting of: β-globin/IgG chimeric intron, β-globin intron, IgG intron, mouse CMV first intron, rat CMV first intron, human CMV first intron, Ig variable region intron and splice acceptor sequence (Bothwell et al., (1981) Cell, 24: 625-637; U.S. Pat. No. 5,024,939), introns of the chicken TNT gene and introns of EF1alpha, preferably the first intron of EF1alpha. In a preferred embodiment, the intron flanking the first exon can be the cTNT intron number 4 (cTNT-I4), the cTNT intron number 5 (cTNT-I5) or the EF1alpha first intron. In more preferred embodiment, the intron flanking the first exon is cTNT-I4.

In order to adjust the ratio of expression between the first and second exons, small variations in the intron upstream of the first exon can be introduced. Such variations comprise altering the number of pyrimidine bases in a polypyrimidine (poly(Y)) tract located upstream of the first exon. As is demonstrated in Example 2, altering the number of pyrimidine bases in the poly(Y) tract can have a major impact on the expression of the first and second exons. For example, increasing the number of pyrimidine bases in the poly(Y) tract strengthens the splice acceptor site of the second exon coding for the second polypeptide. Alternatively, decreasing the number of pyrimidine bases in the poly(Y) tract weakens the splice acceptor site of the first exon coding for the first polypeptide. It was found that decreasing the strength of the first splice acceptor site upstream of the first exon leads towards exclusion of the first exon and therefore results in higher expression from the second exon. In an embodiment of the present invention, the expression construct comprises a poly(Y) tract upstream of the first exon. The number of pyrimidine bases in the poly(Y) tract may comprise between 0 and 30 bases. Preferably the poly(Y) tract comprises a number of pyrimidine bases selected from the group consisting of 28, 27, 26, 25 and 24 bases. More preferably, the poly(Y) tract comprises 10 pyrimidine bases or less, even more preferably 7 bases or less, most preferably 5 bases or less. In one embodiment of the present invention, the poly(Y) tract is absent from the expression construct.

In another embodiment of the present invention, to shift the ratio of expression from the first exon to the second exon, the second splice donor site upstream of the second exon can be eliminated. Such a deletion can be achieved by deleting the exon-intron consensus region and the entire intron upstream of the second splice acceptor region. Such a deletion increased the shift from expression of the first polypeptide to expression of the second polypeptide. In a preferred embodiment, the elimination of the second splice donor site can be combined with a reduction in the number of pyrimidine bases in the poly(Y) tract upstream of the first exon of the expression construct. Combination of these two features led to almost predominant expression of the second exon and therefore the second polypeptide, as demonstrated in Example 1.

In an aspect of the present invention, the ratio of expression between the first and second exons can be altered by using introns of the same sequence to flank the first exon, altering the number of pyrimidine bases in the poly(Y) tract and/or eliminating the splice donor site upstream of the second flanking intron.

In another embodiment of the present invention, the expression construct further comprises a splice donor site and a splice acceptor site that flank an intron downstream of a promoter region at the 5′ end of the expression construct. These constitutive intron, splice donor and splice acceptor sites are constitutively spliced during maturation of the pre-mRNA into mature mRNA. These constitutive components of the expression construct are separated from the intron upstream of the first exon by a 5′untranslated region. In a further embodiment of the present invention, a polyadenylation site is located downstream of the second exon at the 3′ end of the construct.

In an aspect of the present invention, the expression construct is suitable for expressing two or more polypeptides, in particular polypeptide multimers for example antibodies or fragments thereof.

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) which are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies are grouped into classes, also referred to as isotypes, as determined genetically by the constant region. Human constant light chains are classified as kappa (Cκ) and lambda (Cλ) light chains. Heavy chains are classified as mu (μ), delta (δ), gamma (γ), alpha (α), or epsilon (ε), and define the antibody's isotype as IgM, IgD, IgG, IgA, and IgE, respectively. The IgG class is the most commonly used for therapeutic purposes. In humans this class comprises subclasses IgG1, IgG2, IgG3 and IgG4.

The term “Fab” or “Fab region” as used herein includes the polypeptides that comprise the VH, CH1, VL, and CL immunoglobulin domains. Fab may refer to this region in isolation, or this region in the context of a full length antibody or antibody fragment.

The term “Fc” or “Fc region”, as used herein includes the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM. Fc may include the J chain. For IgG, Fc comprises immunoglobulin domains C gamma 2 and C gamma 3 (Cy2 and Cy3) and the hinge between C gamma 1 (Cγ1) and C gamma 2 (Cγ2). Although the boundaries of the Fc region may vary, the human IgG heavy chain Fc region is usually defined to comprise residues C226 or P230 to its carboxyl-terminus, wherein the numbering is according to the EU numbering system. For human IgG1 the Fc region is herein defined to comprise residue P232 to its carboxyl-terminus, wherein the numbering is according to the EU numbering system (Edelman G M et al., (1969) Proc Natl Acad Sci USA, 63(1): 78-85). Fc may refer to this region in isolation or this region in the context of an Fc polypeptide, for example an antibody.

The term “full length antibody” as used herein includes the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains VL and CL, and each heavy chain comprising immunoglobulin domains VH, CH1 (Cγ1), CH2 (Cγ2), and CH3 (Cγ3). In some mammals, for example in camels and llamas. IgG antibodies may consist of only two heavy chains, each heavy chain comprising a variable domain attached to the Fc region.

Antibody fragments include, but are not limited to, (i) the Fab fragment consisting of VL, VH, CL and CH1 domains, including Fab′ and Fab′-SH, (ii) the Fd fragment consisting of the VH and CH1 domains, (iii) the Fv fragment consisting of the VL and VH domains of a single antibody; (iv) the dAb fragment (Ward E S et al., (1989) Nature, 341: 544-546) which consists of a single variable, (v) F(ab′)₂ fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al., (1988) Science 242: 423-426; Huston J S et al., (1988) Proc. Natl. Acad. Sci. USA. 85: 5879-83), (vii) bispecific single chain Fv dimers (PCT/US92/09965), (viii) “diabodies” or “triabodies”, multivalent or multispecific fragments constructed by gene fusion (Tomlinson I & Hollinger P (2000) Methods Enzymol. 326: 461-79; WO94/13804; Holliger P et al., (1993) Proc. Natl. Acad. Sci. USA, 90: 6444-48) and (ix) scFv genetically fused to the same or a different antibody (Coloma M J & Morrison S L (1997) Nature Biotechnology, 15(2): 159-163).

Antibodies and fragment thereof that can be expressed by an expression construct as described herein may bind to an antigen selected from the list consisting of: AXL, Bc12, HER2, HER3, EGF, EGFR, VEGF, VEGFR, IGFR, PD-1, PD-1L, BTLA, CTLA-4, GITR, mTOR, CS1, CD3, CD16, CD16a, CD19, CD20, CD22, CD25, CD27, CD28, CD30, CD32b, CD33, CD38, CD40, CD52, CD64, CD79, CD89, CD137, CD138, CA125, cMet, CCR6, MUCI, PEM antigen, Ep-CAM, EphA2, 17-1a, CEA, AFP, HLA class II, HLA-DR, HSG, IgE, IL-12, IL-17a, IL-18, IL-23, IL-1alpha, IL-1beta, GD2-ganglioside, MCSP, NG2, SK-I antigen, Lag3, PAR2, PDGFR, PSMA, Tim3, TF, CTLA4, TLIA, TIGIT, SIRPa, ICOS, Treml2, NCR3, HVEM, OX40, VLA-2 and 4-1BB.

Bispecific or heterodimeric antibodies have been available in the art for many years. However the generation of such antibodies is often associated with the presence of mispaired by-products, which reduces significantly the production yield of the desired bispecific antibody and requires sophisticated purification procedures to achieve product homogeneity. The mispairing of immunoglobulin heavy chains can be reduced by using several rational design strategies, most of which engineer the antibody heavy chains for heterodimerisation via the design of man-made complementary heterodimeric interfaces between the two subunits of the CH3 domain homodimer. The first report of an engineered CH3 heterodimeric domain pair was made by Carter et al. describing a “protuberance-into-cavity” approach for generating a hetero-dimeric Fc moiety (U.S. Pat. No. 5,807,706; ‘knobs-into-holes’; Merchant A M et al., (1998) Nat Biotechnol, 16(7):677-81). Alternative designs have been recently developed and involved either the design of a new CH3 module pair by modifying the core composition of the modules as described in WO2007110205 or the design of complementary salt bridges between modules as described in WO2007147901 or WO2009089004. The disadvantage of the CH3 engineering strategies is that these techniques still result in the production of a significant amount of undesirable homo-dimers. A more preferred technique for generating bispecific antibodies in which predominantly heterodimers are produced is described in WO2012131555. Bispecific antibodies can be generated to a number of targets, for example, a target located on tumour cells and/or a target located on effector cells. Preferably, a bispecific antibody can bind to two targets selected from the list consisting of: AXL, Bcl2, HER2, HER3, EGF, EGFR, VEGF, VEGFR, IGFR, PD-1, PD-1L, BTLA, CTLA-4, GITR, mTOR, CS1, CD3, CD16, CD16a, CD19, CD20, CD22, CD25, CD27, CD28, CD30, CD32b, CD33, CD38, CD40, CD52, CD64, CD79, CD89, CD137, CD138, CA125, cMet, CCR6, MUCI, PEM antigen, Ep-CAM, EphA2, 17-1a, CEA, AFP, HLA class II, HLA-DR, HSG, IgE, IL-12, IL-17a, IL-18, IL-23, IL-1alpha, IL-1beta, GD2-ganglioside, MCSP, NG2, SK-1 antigen, Lag3, PAR2, PDGFR, PSMA, Tim3, TF, CTLA4, TLIA, TIGIT, SIRPa, ICOS, Treml2, NCR3, HVEM, OX40, VLA-2 and 4-1BB.

In a further aspect, the present invention provides a host cell comprising an expression construct or an expression vector as described supra. The host cell can be a human or non-human cell. Preferred host cells are mammalian cells. Preferred examples of mammalian host cells include, without being restricted to, Human embryonic kidney cells (Graham F L et al., (1977) J. Gen. Virol. 36: 59-74), MRC5 human fibroblasts, 983M human melanoma cells, MDCK canine kidney cells, RF cultured rat lung fibroblasts isolated from Sprague-Dawley rats, B16BL6 murine melanoma cells, P815 murine mastocytoma cells, MT1 A2 murine mammary adenocarcinoma cells, PER:C6 cells (Leiden, Netherlands) and Chinese hamster ovary (CHO) cells or cell lines (Puck T T et al., (1958), J. Exp. Med. 108: 945-955).

In a particular preferred embodiment the host cell is a Chinese hamster ovary (CHO) cell or cell line. Suitable CHO cell lines include e.g. CHO-S (Invitrogen, Carlsbad, Calif., USA), CHO K1 (ATCC CCL-61), CHO pro3-, CHO DG44, CHO P12 or the dhfr-CHO cell line DUK-BII (Urlaub G & Chasin L A (1980) PNAS 77(7): 4216-4220), DUXBI I (Simonsen C C & Levinson A D (1983) PNAS 80(9): 2495-2499), or CHO-K1SV (Lonza, Basel, Switzerland).

In a preferred aspect of the present invention, the optimal ratio of expression of the first polypeptide to the second polypeptide will be determined in transient transfection experiments. The ratio of splicing remains similar in transient and in stable cell lines. The construct with the optimal splice ratio can then be used for stable cell line generation, leading to cell lines that express for example, an antibody heavy and light chain (or all subunits of a bispecific molecule) at an optimal ratio. In an embodiment of the invention, the expression construct permits stable expression at an unchanged ratio for multiple generations, as shown in Example 2. Furthermore, use of a selection pressure is not required to maintain stable expression at the desired ratio.

In one aspect, the splice ratio of antibody heavy chain to light chain for optimal expression may be 1:1. Preferably the splice ratio of antibody heavy chain to light chain for optimal expression may be 1:2 or 1:3 or 2:3. Alternatively, the splice ratio of antibody heavy chain to light chain for optimal expression may be 2:1 or 3:1 or 3:2. Such a ratio for optimal expression will be dependent on the respective antibody.

In a further aspect, for the optimal expression of bispecific antibodies the different subunits may be expressed at different ratios using alternative splicing. A preferred bispecific antibody of the present invention comprises the subunits of a heavy chain, a light chain and an Fc-scFv. For a bispecific antibody, as shown in the present invention, the ratio of heavy chain to Fc-scFv expression was found to be the most important parameter. Therefore the splice ratio of heavy chain to Fc-scFv for optimal expression may be 1:1. Preferably the splice ratio of heavy chain to Fc-scFv for optimal expression may be 1:2 or 1:3 or 2:3. Alternatively, the splice ratio of heavy chain to Fc-scFv for optimal expression may be 2:1 or 3:1 or 3:2. Such a ratio for optimal expression will be dependent on the respective antibody.

In a further aspect, the present disclosure provides an in vitro method for the expression of a polypeptide, comprising transfecting a host cell with the expression construct or an expression vector as described supra culturing the host cell and recovering the polypeptide. The polypeptide is preferably a heterologous, more preferably a human polypeptide.

For transfecting the expression construct or the expression vector into a host cell according to the present invention any transfection technique such as those well-known in the art, e.g. electoporation, calcium phosphate co-precipitation, DEAE-dextran transfection, lipofection, can be employed if appropriate for a given host cell type. It is to be noted that the host cell transfected with the expression construct or the expression vector of the present invention is to be construed as being a transiently or stably transfected cell line. Thus, according to the present invention the present expression construct or the expression vector can be maintained episomally i.e. transiently transfected or can be stably integrated in the genome of the host cell i.e. stably transfected.

A transient transfection is characterised by non-appliance of any selection pressure for a vector borne selection marker. In transient expression experiments which commonly last two to up to ten days post transfection, the transfected expression construct or expression vector are maintained as episomal elements and are not yet integrated into the genome. That is the transfected DNA does not usually integrate into the host cell genome. The host cells tend to lose the transfected DNA and overgrow transfected cells in the population upon culture of the transiently transfected cell pool. Therefore expression is strongest in the period immediately following transfection and decreases with time. Preferably, a transient transfectant according to the present invention is understood as a cell that is maintained in cell culture in the absence of selection pressure up to a time of two to ten days post transfection.

In a preferred embodiment of the invention the host cell e.g. the CHO host cell is stably transfected with the expression construct or the expression vector of the present invention. Stable transfection means that newly introduced foreign DNA such as vector DNA is becoming incorporated into genomic DNA, usually by random, non-homologous recombination events. The copy number of the vector DNA and concomitantly the amount of the gene product can be increased by selecting cell lines in which the vector sequences have been amplified after integration into the DNA of the host cell. Therefore, it is possible that such stable integration gives rise, upon exposure to further increases in selection pressure for gene amplification, to double minute chromosomes in CHO cells. Furthermore, a stable transfection may result in loss of vector sequence parts not directly related to expression of the recombinant gene product, such as e.g. bacterial copy number control regions rendered superfluous upon genomic integration. Therefore, a transfected host cell has integrated at least part or different parts of the expression construct or the expression vector into the genome.

In a further aspect, the present disclosure provides the use of the expression construct or an expression vector as described supra for the expression of a heterologous polypeptide from a mammalian host cell, in particular the use of the expression construct or an expression vector as described supra for the in vitro expression of a heterologous polypeptide from a mammalian host cell.

An expression construct as described in the present invention can be used in a method of optimizing the expression level of a protein of interest. For example, when the protein of interest is an antibody, the expression ratio of the light chain to the heavy chain or vice versa can be altered, to achieve the optimal expression level of the antibody when expressed in a host cell. Using an expression construct comprising in a 5′ to 3′ direction:

a promoter,

an optional first splice donor site;

a first flanking intron;

a splice acceptor site;

a first exon encoding a first polypeptide;

an optional second splice donor site;

a second flanking intron;

a splice acceptor site; and

a second exon encoding a second polypeptide,

the expression level of a protein of interest may be optimised by a method comprising the steps of:
(i) using first and second flanking introns having a nucleic acid sequence homology of at least 80% for a stretch of nucleic acids of at least 50 nucleotides;
(ii) reducing the number of pyrimidine bases in a poly(Y) tract located upstream of the first exon or increasing the number of pyrimidine bases in a poly(Y) tract located downstream of the first exon; and/or
(iii) deleting the splice donor site upstream of the second flanking intron.

Furthermore, an expression construct as described in the present invention can be used in a method of optimizing the heterodimerisation level of a protein of interest. For example, if the protein of interest is a bispecific antibody, such a bispecific antibody may be encoded by one or more expression constructs according to the present invention, which encode a heavy chain, light chain and Fc-scFv. By using the methods of alternative splicing as described herein, the expression ratio of the heavy chain to Fv-scFv or vice versa, for example, can be altered to achieve the optimal expression level of the bispecific antibody when expressed in a host cell. Using an expression construct comprising in a 5′ to 3′ direction:

a promoter,

an optional first splice donor site;

a first flanking intron;

a splice acceptor site;

a first exon encoding a first polypeptide;

an optional second splice donor site;

a second flanking intron;

a splice acceptor site; and

a second exon encoding a second polypeptide,

the heterodimerisation level of a protein of interest may be optimised by a method comprising the steps of:
(iv) using first and second flanking introns having a nucleic acid sequence homology of at least 80% for a stretch of nucleic acids of at least 50 nucleotides;
(v) reducing the number of pyrimidine bases in a poly(Y) tract upstream of the first exon or increasing the number of pyrimidine bases in a poly(Y) tract downstream of the first exon; and/or
(vi) deleting the splice donor site upstream of the second flanking intron.

Expression and recovering of the protein can be carried out according to methods known to the person skilled in the art.

In a further aspect, the present disclosure provides the use of the expression construct or the expression vector as described supra for the preparation of a medicament for the treatment of a disorder.

In a further aspect, the present disclosure provides the expression construct or the expression vector as described supra for use as a medicament for the treatment of a disorder.

In a further aspect, the present disclosure provides the expression construct or the expression vector as described supra for use in gene therapy.

### EXAMPLES

### Example 1

### Materials and Methods

### LB Culture Plates

500 ml of water was mixed and boiled with 16 g of LB Agar (Invitrogen, Carlsbad, Calif., USA) (1 liter of LB contains 10 g tryptone, 5 g yeast extract and 10 g NaCl). After cooling, the respective antibiotic was added to the solution which was then distributed in culture dishes (ampicilin plates at 100 μg/ml and kanamycin plates at 50 μg/ml).

### Polymerase Chain Reaction (PCR)

All PCRs were performed using 1 μl of dNTPs (10 mM for each dNTP; Invitrogen, Carlsbad. Calif., USA), 2 units of Phusion® DNA Polymerase (Finnzymes Oy, Espoo, Finland), 25 nmol of Primer A (Mycrosynth, Balgach, Switzerland), 25 nmol of Primer B (Mycrosynth, Balgach, Switzerland), 10 μl of 5×HF buffer (7.5 mM MgCl2, Finnzymes, Espoo, Finland), 1.5 μl of Dimethyl sulfoxide (DMSO, Finnzymes, Espoo, Finland) and 1-3 μl of the template (10-20 ng) in a 50 μl final volume.

The PCRs were started by an initial denaturation at 98° C. for 3 minutes, followed by 35 cycles of 30 sec denaturation at 98° C., 30 sec annealing at a primer-specific temperature (according to CG content) and elongation at 72° C. (30 sec/kB of template). A final elongation at 72° C. for 10 min was performed before cooling and keeping at 4° C. All primers used for this example are listed in the following Table 1.

TABLE 1
List of all primers used for cloning

| | Seq ID | |
|---|---|---|
| Primer | No: | Sequence |
| Glnpr991 | 001 | GGTCATTTCGAATCATTACTTGTACAGCTCGT |
| Glnpr1095 | 002 | CGCTGGCTAGCGTTTAAACTTAAG |
| Glnpr1096 | 003 | ATCGTTCGAATATGGGCCCTCTCGCACACCGGTCTCCTCTTCCTCCTC |
| Glnpr1097 | 004 | TATAGGGCCCTGTGAGCAAGGGCGAGGAG |
| Glnpr1098 | 005 | GCGCTTCGAATCATTACTTGTACAGCTCGTC |
| Glnpr1099 | 006 | TATAGGGCCCTCTACAGGAACAGGTGGTG |
| Glnpr1100 | 007 | ATTAACCGGTGCCTCCTCCGAGGACGTC |
| Glnpr1138 | 008 | AATTAAGCTAGCGTTTAAACTTAAGCTTCCTTGGATTACAAGGATGACGAT |
| Glnpr1139 | 009 | GTGGCGATATCGCCTGGATCCTGAG |
| Glnpr1140 | 010 | CCAGGCGATATCGCCACCATGGGTGCCTCCTCCGAGGA |
| Glnpr1141 | 011 | CTACCTGAATTCTTCCGTTACTACAGGAACAGGTGGTGGCGGC |
| Glnpr1142 | 012 | GAGGAGACCGGTGCCACCATGGAGCAAGGGCGAGGAGCTGT |
| Glnpr1158 | 013 | AATTAAGCTAGCGTTTAAACTTAAGCTTCCTTGGAGGACCCAGTAC |
| | | CCGGATCTAGAGGTAGG |
| Glnpr1180 | 014 | AATTAAACCGGTGCCACCATGGTGAGCAAGGGCGAGGAGC |
| Glnpr1181 | 015 | GCGCGGCTAGCGTTTAAACTTAAGC |
| Glnpr1182 | 016 | TTGTGATATCGCCTGGATCCTGTGCAATAAGGACAGGGTTAGCCAG |
| | | GTGCCTTAAAGCTGTG |
| Glnpr1183 | 017 | AGCAGGATATCGCCTGGATCCTGAGACAGGGAGGAGG |
| Glnpr1184 | 018 | ATATGATATCGCCTGGATCCTGAGCCAGGGAGCAGGCAAGGCAAG |
| | | AAGCGCAGAGGTTAGCC |
| Glnpr1185 | 019 | AGTCGATATCGCCTGGATCCTGAGCCAGGTAGCAGGGAAGGGAAG |
| Glnpr1186 | 020 | GATGGATATCGCCTGGATCCTGAGCCAGGGAGGAGGGAAGGCAAC |
| | | AAGCGCAGAGGTTAGCC |
| Glnpr1187 | 021 | GCGCGAATTCAGGTAGTTACTGCAC |
| Glnpr1189 | 022 | TATAACCGGTCTCCTCTTCCTCCTCGTCCTCCTGATCCTCCTGACCT |
| | | GAGCCAGGGAGGAGGGAAG |
| Glnpr1190 | 023 | TAATACCGGTCTCCTCTTCCTCCTCGTCCTCCTGATCCTCCTGACCT |
| | | GAGCCAGGGAGCAGGCAAGGCAAGAAG |
| Glnpr1191 | 024 | ATATACCGGTCTCCTCTTCCTCCTCGTCCTCCTGATCCTCCTGACCT |
| | | GAGACAGGGAGGAGGGAAG |
| Glnpr1192 | 025 | ATATACCGGTCTCCTCTTCCTCCTCGTCCTCCTGATCCTCCTGACCT |
| | | GAGCCAGGGAGGAGGGAAG |
| Glnpr1193 | 026 | ATATACCGGTCTCCTCTTCCTCCTCGTCCTCCTGATCCTCCTGACCT |
| | | GAGCCAGGTAGCAGGGAAGGGAAGAAG |
| Glnpr1237 | 027 | GGCGGCTAGCGTTTAAACTTAAGCTTCCTTGGAGGACCCAGTACCC |
| | | GGATCTAGAGTAGTTACTGCACCTTTCTTTG |
| Glnpr1238 | 028 | ATCGGATATCGCCTGGATCCTGTGCAATAAGGACAGGGTC |
| Glnpr1239 | 029 | GTGGCGATATCGCCTGGATCCTHTGCAATAAGGAC |
| Glnpr1240 | 030 | TGGCGATATCGCCTGGATCCTGTGCAATAAGGACAGCCTTAGCCAG |
| | | GTGCCTTAAAG |
| Glnpr1241 | 031 | TGGCGATATCGCCTGGATCCTGTGCAATAAGGACAGGGTTCTCCAG |
| | | GTGCCTTAAAG |
| Glnpr1242 | 032 | TGGCGATATCGCCTGGATCCTGTGCAATAAGGACAGGGCAAGCCA |
| | | GGTGCCTTAAAG |
| Glnpr1243 | 033 | TGGCGATATCGCCTGGATCCTGTGCAATAAGGACAGCGTAGGCCAG |
| | | GTGCCTTAAAG |
| Glnpr1244 | 034 | GCGATATCGCCTGGATCCTGTCCCCTAAGGACTCGGTTAGCCAGGT |
| | | GCCTTAAAGCTGTG |
| Glnpr1245 | 035 | GCGATATCGCCTGGATCCTGTGCAATCCTCCCAGGGTTAGCCAGGT |
| | | GCCTTAAAGCTGTG |
| Glnpr1246 | 036 | GCGATATCGCCTGGATCCTGTTCCCTCCTCCCTCGGTTAGCCAGGTG |
| | | CCTTAAAGCTGTG |
| Glnpr1285 | 037 | CGGAAGAATTCAGCCACAGCTTTAAGGCACCTGGCTAAC |

### Restriction Digest

For all restriction digests 1 μg of plasmid DNA (quantified with Nano Drop) was mixed to 10-20 units of each enzyme, 4 μl of corresponding 10× NEBuffer (NEB, Ipswich, Mass. USA), and the volume was completed to 40 μl with sterile H₂O. Without further indication, digestions were incubated 1 hour at 37° C. After each preparative digestion of backbone, 1 unit of Calf Intestinal Alkaline Phosphatase (CIP; NEB, Ipswich, Mass., USA) was added and the mix was incubated 30 min at 37° C.

### PCR Purification and Gel Agarose Electrophoresis

To allow digestion all PCR fragments were cleaned prior to restriction digests using the Macherey Nagel NucleoSpin Extract II kit (Macherey Nagel, Oensingen, Switzerland) following the manual of the manufacturer. This protocol was also used for changing buffers of DNA samples.

For gel electrophoresis, 1% gels were prepared using UltraPure™ Agarose (Invitrogen, Carlsbad. Calif., USA) and 50× Tris Acetic Acid EDTA buffer (TAE, pH 8.3; Bio RAD, Munich, Germany). For staining of DNA 1 μl of Gel Red Dye (Biotum, Hayward, Calif., USA) was added to 100 ml of agarose gel. As a size marker 2 μg of the 1 kb DNA ladder (NEB, Ipswich, Mass., USA) was used. The electrophoresis was run for 1 hour at 125 Volts.

The bands of interests were cut out from the agarose gel and purified using the kit NucleoSpin Extract II (Macherey-Nagel, Oensingen, Switzerland), following the manual of the manufacturer.

### Ligation

For each ligation, 4 μl of insert were mixed to 1 μl of vector, 400 units of ligase (T4 DNA ligase. NEB, Ipswich, Mass., USA), 1 μl of 10× ligase buffer (T4 DNA ligase buffer; NEB, Ipswich, Mass., USA) in a 10 μl volume. The mix was incubated for 1-2 h at RT.

25-50 μl of competent bacteria (One Shot® TOP 10 Competent E. coli; Invitrogen, Carlsbad, Calif., USA) were thawed on ice for 5 minutes. 5 μl of ligation product were added to competent bacteria and incubated for 20-30 min on ice before the thermic shock for 1 minute at 42° C. Then, 500 μl of S.O.C medium (Invitrogen, Carlsbad, Calif., USA) were added per tube and incubated for 1 hour at 37° C. under agitation with 600 rpm on thermoshaker. Finally, the bacteria were put on a LB plate with ampicillin (Sigma-Aldrich, St. Louis, Mo., USA) or kanamycin and incubated overnight at 37° C.

### Plasmid Preparation in Small (Mini) and Medium Scale (Midi)

For mini-preparation, colonies of transformed bacteria were grown for 6-16 hours in 2.5 ml of LB and ampicillin or kanamycin at 37° C., 200 rpm. The DNA was extracted with a plasmid purification kit for E. coli (NucleoSpin QuickPure or NucleoSpin Plasmid (No Lid), Macherey Nagel, Oensingen, Switzerland), following the provided manual.

For midi-preparation, transformed bacteria were grown at 37° C. overnight in 200 ml of LB and ampicillin (or kanamycin). Then, the culture was centrifuged 20 min at 725 g and the plasmid was purified using a commercial kit (NucleoBond Xtra Midi; Macherey Nagel, Oensingen, Switzerland) following the protocol provided in the manual of the manufacturer.

Plasmid-DNA from midi-preparation was quantified three times with the Nano Drop ND-1000 Spectrophotometer, confirmed by restriction digest and finally sent for sequencing (Fasteris SA. Geneva, Switzerland).

### Cultivation and Transfection of Cells

The cells were cultivated for routine passaging in 100 ml growth medium (PowerCHO2 (Lonza, Verviers, Belgium), 4 mM Gln for CHO-S cells and Ex-cell293 (Sigma-Aldrich, St. Louis, Mo.), 4 mM Gln for HEK293 cells). Cells were seeded at 0.5E6 cells/ml twice a week and incubated in a shaken incubator in an atmosphere of 5% CO2 and 80% humidity.

The constructs were transfected in CHO-S cells and HEK293 cells. For transfection, the cells were seeded at a density of 1E6 cells/ml prior to the day of transfection. The day of transfection, the cells were resuspended in either Optimem (CHO-S) or RPMI (HEK293) and transfected with JetPEP™ (Polyplus-transfection, Strasbourg, France) according to the manual of the manufacturer. After 5 hours one volume of the respective growth medium was added (for HEK293 cells this was supplemented with Pluronic F68). The cells were analysed three to five days after transfection by FACS for GFP and dsRED expression. The transfection was done in 12 or 24 well plates (TPP, Trasadingen, Switzerland) using a final volume of 2 ml or 1 ml, respectively, or in 50 ml bioreactor tubes (“Tubespins”, TPP) using a final medium volume of 10 ml.

### FACS Analysis

The cells were gated on living cells using forward and side scatter. For the analysis of the ratio of dsRED and GFP expressing cells, compensation was performed using dsRED transfected cells and GFP transfected cells. For the estimation of the shift from dsRED to GFP expressing cells, non-transfected cells were excluded by adding a gate.

### Results

### Design of Constructs and Cloning Steps

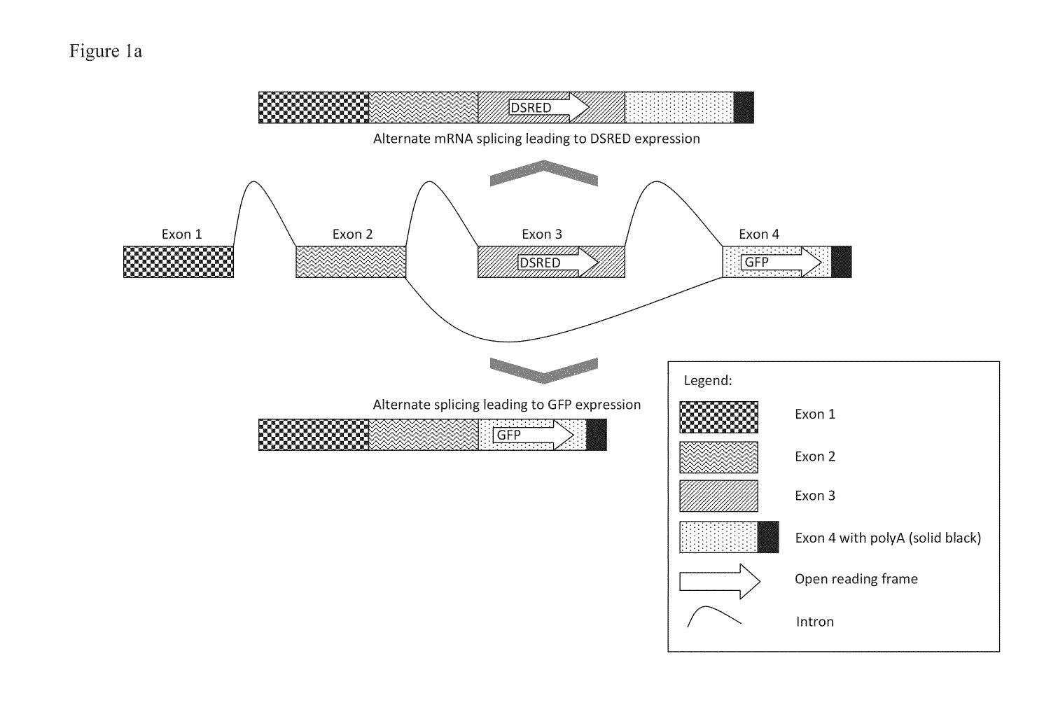

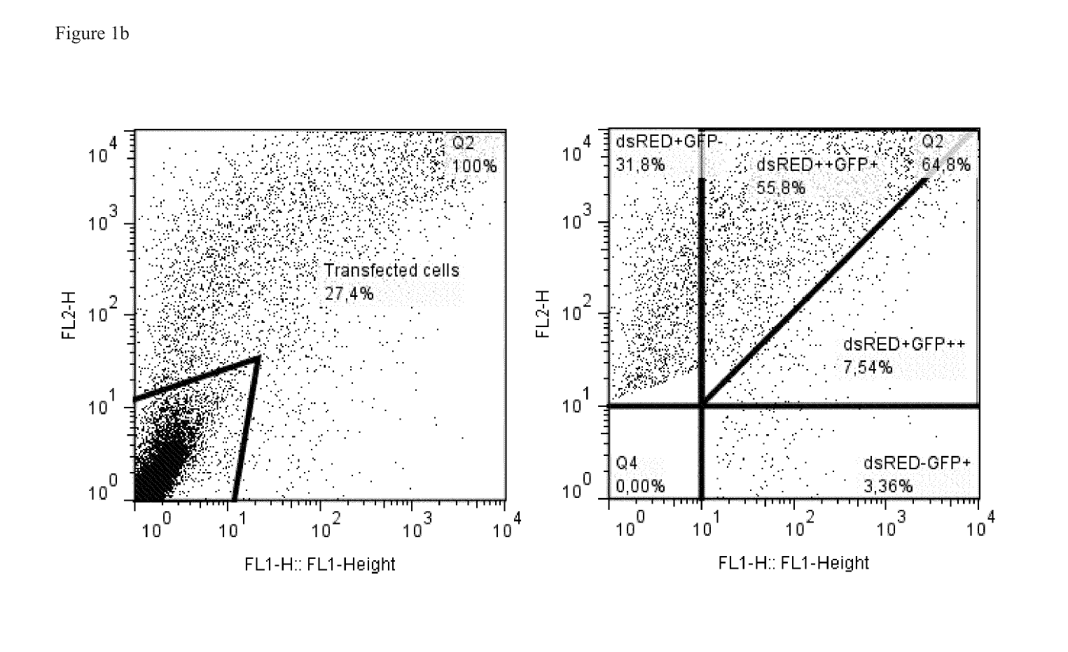

In order to be able to visualize the expression of two alternate open reading frames located on two different exons of the same primary transcript, the fluorescence markers GFP and dsRED were used. Both proteins can be intracellularly expressed at high levels, are well tolerated by cells and can be easily distinguished in FACS analysis or under a fluorescent microscope. A disadvantage of using fluorescent markers is the fact that the measured fluorescence cannot be easily attributed to a quantity of protein and therefore only conclusions on relative expression levels of one protein compared to another are possible. Therefore at this early experimental phase, different constructs were created in order to obtain a range of different relative expression levels from exon 1 and 2 (see scheme in FIG. 1a).

The alternate splicing constructs were made based on the chicken troponin (cTNT) introns 4 and 5 surrounding the alternate cTNT exon 5. Troponin is expressed exclusively in cardiac muscle and embryonic skeletal muscle. Over 90% of the mRNAs include the exon in early embryonic heart and skeletal muscle, whereas >95% of mRNAs in the adult exclude the exon (Cooper & Ordahl (1985) JBC 260(20):11140-8). In the constructs of the present invention, the cTNT introns were cloned as second and third intron of the primary transcript. The first intron is a constitutive intron that is used in combination with the mCMV or the hCMV promoter. It is important to note, that the cTNT intron names used in this example designate an intron sequence and not the position of the intron in the construct (cTNT intron 4 may be intron number 2 or 3 in the constructs). In order to avoid confusion the cTNT intron 4 will be abbreviated cTNT-I4 and the cTNT intron 5 will be abbreviated cTNT-I5, while the position of the introns in the respective construct will be counted using AS intron numbers (for example in the basic construct, cTNT-I4 was cloned in position AS intron #2). In the basal construct (GSC2250), the intron sequences cTNT-I4 (AS intron #2) and cTNT-I5 (AS intron #3) flank a modified alternate exon which contains the open reading frame coding for dsRED. Downstream of AS intron #3 (in basal construct cTNT-I5) follows the exon which contains the open reading of GFP (see FIG. 1a for a schematic drawing).

### Cloning of the Vector Described in Orengo et al

The alternate splicing construct of the invention was based on a construct described by Orengo et al (Orengo J R et al., (2006) Nucleic Acids Res. 2006; 34(22): e148). In this construct, the start codon of the expression cassette is shared between the open reading frames coding for dsRED and GFP, followed by a flag tag and a short nuclear localization sequence. The very short alternate exon flanked by the chicken troponin introns 4 and 5 had been adjusted in length by the authors to be excluded at approximately 50%. If excluded, the open reading of dsRED is in frame with the start codon and only dsRED is expressed. Inclusion of the small alternate exon will introduce a frameshift to the reading frame. The open reading frame of dsRED will be read in the second frame (no stop codon is present in this frame of dsRED) leading to a fusion protein of dsRED (read in the second frame) and GFP. The disadvantages of this technology are numerous. First, one of the proteins is necessarily a fusion protein of the second frame of the first protein and the second protein. Second, not many proteins have a second open reading frame without stop codons and very few proteins will show biological activity with a nonsense protein fused to the N-terminus. Furthermore, this technology is unsuitable for use in a therapeutic context, because of the immunogenic potential of the unfolded fusion protein, therefore this construct was used as a control for the alternate expression of dsRED and GFP and as a basis for further and optimized constructs.

The DNA construct was ordered from GeneArt (Regensburg, Germany, now Life Technologies). The lyophilized plasmid DNA from GeneArt was resuspended according to the specifications of GeneArt and used as template for a PCR amplification using the primers GlnPr1095 and GlnPr1096. This added a NheI site to the 5′ end. The SacII restriction site at the 3′ end was replaced by ApaI and an additional BstBI site was added to the 3′ end. The digestion of this fragment with the restrictions enzymes NheI and BstB allowed ligation into the backbone of pGLEX3HM-MCS, opened using the same enzymes and CIPed. The pGLEX3HM-MCS vector contains an expression cassette under control of the hCMV promoter. The new vector with the GeneArt fragment in the pGLEX3HM-MCS backbone was called pGLEX3-ASC.

EGFP was amplified from pGLEX3 (a vector previously cloned in-house that contained an open reading frame coding for EGFP (in short: GFP) derived from the plasmid pEGFP-N1 (Clontech)) using the primers GlnPr1097 and GlnPr1098. The amplification removes the start codon ATG from the open reading frame of GFP and adds an ApaI site to the 5′ end and a BstBI site to the 3′ end. Digestion of the amplicon using the restriction enzymes ApaI, BstBI and ligation into pGLEX3-ASC, opened with the same enzymes, led to the vector pGLEX3-ASC-GFP.

The dsRED open reading frame was amplified from the plasmid pdsRED-Express 1 (Clontech) using the primers GlnPr1099 and GlnPr1100. These primers remove the start codon ATG from the 5′ end and add an AgeI restriction site to the 5′ end and an ApaI site to the 3′ end. The amplicon was digested using the restriction enzymes AgeI and ApaI and ligated in pGLEX3-ASC-GFP, digested using the same enzymes and CIPed. This generated plasmid pGLEX3-ASC-dsRED-GFP. This vector contains the construct created by Orengo et al., supra.

Cloning of Vector pGLEX3-ASC-dsRED-GFP-woFLAGcorr

The modification of the alternate splicing construct was done by modifying PCR. A first PCR was performed using the primers GlnPr1142 and GlnPr991 and the template pGLEX3-ASC-dsRED-EGFP. The PCR product was cut using the restriction enzymes AgeI and BstBI and cloned into pGLEX-ASC-dsRED-GFP opened using the same enzymes and CIPed, leading to the intermediate construct pGLEX-ASC-dsRED-GFP-interm. Using the plasmid pGLEX3-ASC-dsRED-EGFP as template, a second amplicon was obtained using primers GlnPr1138 and GlnPr1139 and a third using primers GlnPr1140 and GlnPr1141. These two amplicons were then used as templates for a fusion PCR using primers GlnPr1138 and GlnPr1141.

This fusion product was cut using the restriction enzymes NheI and EcoRI and cloned into the vector pGLEX-ASC-dsRED-GFP-interm opened with the same enzymes and CIPed in order to obtain the final construct pGLEX3-ASC-dsRED-GFP-sep. This vector was numbered GSD634.

The flag tag still present in pGLEX3-ASC-dsRED-GFP-sep contains the sequence motif ATG that might be used as a translation start point (start codon). The deletion was done by modifying PCR, using the primers GlnPr1158 and 1139 and plasmid GSD634 as template. The PCR product was digested using the restriction enzymes NheI and EcoRV and cloned into GSD634, opened using the same enzymes followed by a CIP treatment in order to minimize re-circularisation. The resulting plasmid was called pGLEX3-ASC-dsRED-GFP-sepwoFLAG with the batch number GSC2223 (SEQ ID No: 110). The resulting midi scale preparation of this plasmid received the batch number GSD679 and has the same sequence as GSC2223.

It was observed that two nucleotides of the GFP had been different compared to the standard GFP sequence. This was due to the design of a forward primer. Using the primers GlnPr991 and 1180 and the template pGLEX3, the GFP fragment was re-amplified with the correct sequence. This fragment was digested using the enzyme AgeI and cloned into the vector the backbone of GSD679, opened using AgeI and subsequently CIPed, leading to the vector pGLEX3-ASC-dsRED-GFP-woFLAGcorr. The miniprep of pGLEX3-ASC-dsRED-GFP-woFLAGcorr was given the batch number GSC2246 and the midiprep, the batch number GSC2250 (SEQ ID No: 38), therefore both these constructs had the same sequence.

Cloning of Constructs with Alternate Splicing Pattern

The construct GSC2250 was further modified in order to obtain constructs with a different ratio of alternative splicing, leading to a shift in expression from the first to the second open reading frame in the construct. The modifications were introduced by amplification of the chicken troponin intron 4 or 5 using modified primers. These amplicons were then recloned in the backbone of GSC2250 or a similar plasmid using the restriction enzymes NheI and EcoRV for cloning in position of the AS intron #2 and EcoRI and AgeI for cloning in the position of the AS intron #3 (see FIG. 1 for orientation). The following Table 2 and Table 3 summarize the primers and the templates used for the necessary cloning steps of the introns in position AS intron #2 and 3, respectively. Table 4 shows all combinations that were cloned.

TABLE 2
Primers and templates used for the modifications of the AS intron #2.

| Name of | Forward | Backward | Template used for |
|---|---|---|---|
| construct | primer used | primer used | amplification |
| I4 (22 + 1) | GlnPr1181 | GlnPr1183 | GSC2246 (miniprep) |
| I4 (15Y-5′) | GlnPr1181 | GlnPr1186 | GSC2246 (miniprep) |
| I4 (15Y-3′) | GlnPr1181 | GlnPr1185 | GSC2246 (miniprep) |
| I4 (22Y-3) | GlnPr1181 | GlnPr1184 | GSC2246 (miniprep) |
| I4 (5Y) | GlnPr1181 | GlnPr1182 | GSC2246 (miniprep) |
| I4 (5Y-5) | GlnPr1181 | GlnPr1245 | GSC2338 |
| I4 (0Y) | GlnPr1181 | GlnPr1246 | GSC2338 |
| I4 (5Ynude) | GlnPr1181 | GlnPr1244 | GSC2338 |
| I4 (5Y, b-2) | GlnPr1181 | GlnPr1243 | GSC2338 |
| I4 (5Y, b-a) | GlnPr1181 | GlnPr1242 | GSC2338 |
| I4 (5Y, b-ct | GlnPr1181 | GlnPr1241 | GSC2338 |
| I4 (5Y, b-y) | GlnPr1181 | GlnPr1240 | GSC2338 |
| I4 (5Y-G) | GlnPr1181 | GlnPr1239 | GSC2338 |
| cTNT-I5 | GlnPr1237 | GlnPr1238 | GSC2250 |

TABLE 3
Primers and templates used for the modifications of the AS intron #3

| Name of | Forward | Backward | Template used for |
|---|---|---|---|
| construct | primer used | primer used | amplification |
| I5 (22Y + 1) | GlnPr1187 | GlnPr1191 | Amplicon 1187/1188 |
| | | | on GSC2246 (miniprep) |
| I5 (22Y-3) | GlnPr1187 | GlnPr1190 | Amplicon 1187/1188 |
| | | | on GSC2246 (miniprep) |
| I5 (22Y) | GlnPr1187 | GlnPr1189 | Amplicon 1187/1188 |
| | | | on GSC2246 (miniprep) |
| I5 (15Y-3′) | GlnPr1187 | GlnPr1193 | Amplicon 1187/1188 |
| | | | on GSC2246 (miniprep) |
| I5 (15Y-5′) | GlnPr1187 | GlnPr1192 | Amplicon 1187/1188 |
| | | | on GSC2246 (miniprep) |
| I4 (sh) | GlnPr1285 | GlnPr991 | GSC2741 |

Screening of Alternate Splicing Constructs in Transient Using GFP and dsRED

The different constructs were cloned in the combinations listed in Table 4, produced at midi scale and thoroughly verified by sequencing (Fasteris, Plan-les-Ouates, Switzerland). An alignment of all introduced modifications is shown in FIG. 2. The plasmids were transfected in CHO-S and in HEK293 cells. As a positive control, vectors expressing only dsRED (GSD636, an in-house vector based on pGLEX3 expressing the dsRED gene, derived from pDsRED-Express 1 (Clontech)) and GFP (pEGFP-N1, Clontech) were transfected into the host cells, respectively. The analysis was done by flow cytometry, supported by fluorescence microscopy using adequate filters.

The transfections were done in 12 well plate scale as described in the material and methods part using HEK293 and CHO-S cells. Although this transfection scale is robust, variations in the transfection efficiency do not allow conclusions on the absolute expression level of the individual constructs.

TABLE 4
List of constructs used in order to shift the splice ratio from the first exon (dsRED expression) to the second exon
(GFP expression). Available clones are indicated by the in-house plasmid batch number and the SEQ ID listing. The
SEQ ID comprises the entire mRNA, from the nucleotide of the first exon to the end of the SV 40 poly(A) site.

| | | | Intron constructs used upstream of the alternate exon | | | | | | |
|---|---|---|---|---|---|---|---|---|---|
| | | | (position AS intron #3) | | | | | | |
| | | Name of | cTNT- | I5 | I5 | I5 | I5 | cTNT- | I4 |
| | | construct | I5 | (22Y + 1) | (22Y − 3) | (22Y) | (15Y − 3′) | I4 | (sh) |
| Intron constructs used | Poly(Y) tract | cTNT-I4 | GSC 2250 | GSC 2329 | | GSC 2330 | GSC 2323 | GSC 2619 | GSC 2781 |
| upstream of the alter- | modifications | | Seq ID 38 | Seq ID 39 | | Seq ID 40 | Seq ID 41 | Seq ID 42 | Seq ID 43 |
| nate exon (position | | I4 | GSC 2342 | | GSC 2328 | GSC 2321 | GSC 2324 | | |
| AS intron #2) | | (22Y + 1) | Seq ID 44 | | Seq ID 45 | Seq ID 46 | Seq ID 47 | | |
| | | I4 | GSC 2339 | | | GSC 2334 | GSC 2336 | | |
| | | (15Y − 5′) | Seq ID 48 | | | Seq ID 49 | Seq ID 50 | | |
| | | I4 | GSC 2340 | GSC 2331 | GSC 2453 | GSC 2325 | GSC 2332 | | |
| | | (15Y − 3′) | Seq ID 51 | Seq ID 52 | Seq ID 53 | Seq ID 54 | Seq ID 55 | | |
| | | I4 | GSC 2341 | | GSC 2326 | GSC 2454 | GSC 2327 | | |
| | | (22Y − 3) | Seq ID 56 | | Seq ID 57 | Seq ID 58 | Seq ID 59 | | |
| | | I4 | GSC 2338 | GSC 2335 | GSC 2333 | GSC 2337 | GSC 2322 | | |
| | | (5Y) | Seq ID 60 | Seq ID 61 | Seq ID 62 | Seq ID 63 | Seq ID 64 | | |
| | | I4 | | | | GSC 2617 | | GSC 2739 | GSC 2782 |
| | | (5Y − 5) | | | | Seq ID 65 | | Seq ID 66 | Seq ID 67 |
| | | I4 | | | | GSC 2621 | | GSC 2740 | GSC 2783 |
| | | (0Y) | | | | Seq ID 68 | | Seq ID 69 | Seq ID 70 |
| | | I4 | | | | GSC 2622 | | GSC 2742 | GSC 2784 |
| | | (5Ynude) | | | | Seq ID 71 | | Seq ID 72 | Seq ID 73 |
| | Branch point | I4 | | | | GSC 2620 | | GSC 2737 | |
| | mutations | (5Y, b − 2) | | | | Seq ID 74 | | Seq ID 75 | |
| | | I4 | | | | | | GSC 2743 | |
| | | (5Y, b − a) | | | | | | Seq ID 77 | |
| | | I4 | | | | GSC 2615 | | GSC 2738 | |
| | | (5Y, b − ct) | | | | Seq ID 76 | | Seq ID 78 | |
| | | I4 | | | | GSC 2618 | | GSC 2975 | |
| | | (5Y, b − y) | | | | Seq ID 79 | | Seq ID 80 | |
| | Intron- | I4 | | | | GSC 2613 | | | |
| | Exon | (5Y, G) | | | | Seq ID 81 | | | |
| | consensus | | | | | | | | |
| | Intron | cTNT-I5 | GSC 2614 | | | | | GSC 2741 | GSC 2780 |
| | Switch | | Seq ID 82 | | | | | Seq ID 83 | Seq ID 84 |

Expression of Constructs with Modifications in the Poly(Y) Tract

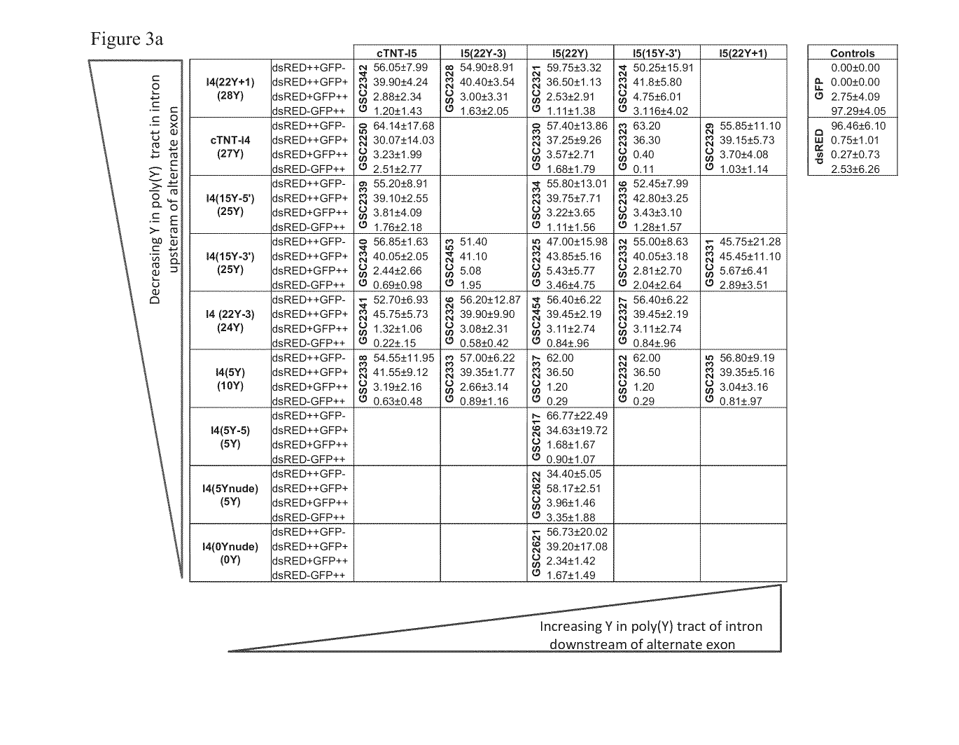

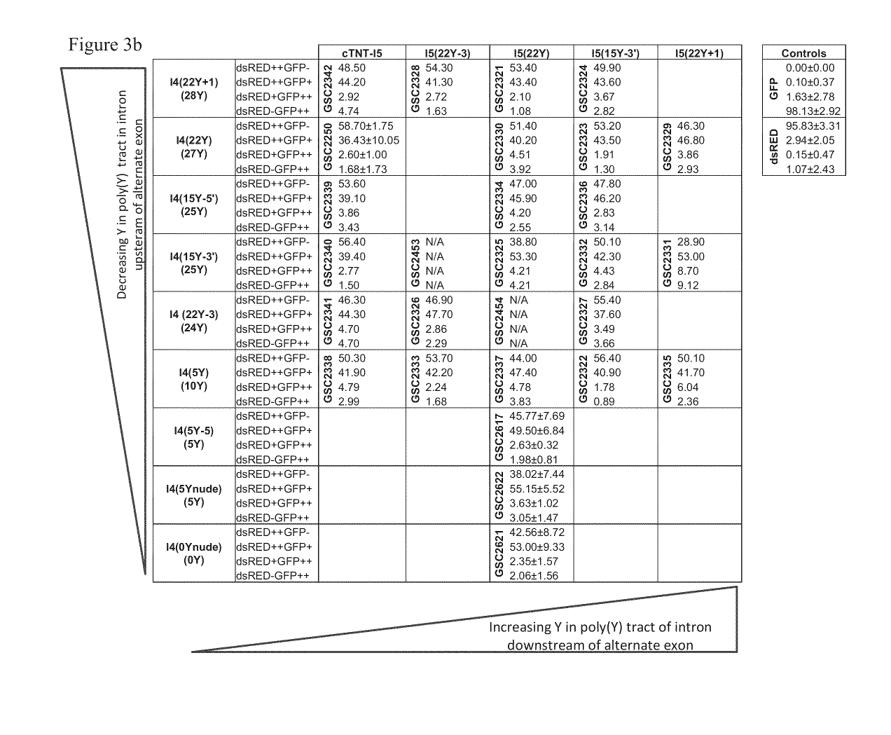

The basal construct GSC2250 contains the alternate exon coding for the open reading frame of dsRED flanked by the unmodified cTNT-I4 sequence as AS intron #2 and the unmodified cTNT-I5 sequence as AS intron #3, followed by an exon coding for the open reading frame of GFP (orientation in short cTNT-I4|cTNT-I5). In transfected CHO-S or HEK293 cells, the construct shows expression of dsRED and GFP (see FIG. 3). This confirmed that the construct leads to alternate splicing. Nevertheless, dsRED expression was largely favoured over GFP expression (see FIGS. 3a & b). The splice acceptor site of the alternate exon coding for dsRED is competing with the second splice acceptor site of the exon coding for GFP. It has been shown that the abundance of Ys (the pyrimidine bases C or T) between the branch point and the intron-exon border (the so called poly(Y) tract) is important for the strength of the splice acceptor site (see, for example, Dominiski & Kole (1992) Mol Cell Biol 12(5): 2108-14). A reduction of the splice acceptor strength by reducing the amount of Ys was expected to lead to preferred exclusion of the alternate exon coding for dsRED and therefore eventually to more expression of GFP.

Different constructs with decreasing amount of Ys (from 28 in a modified version of the basic construct cTNT-I4 down to 0) in the poly(Y) tract (see FIG. 2a for an alignment) of cTNT-I4 in position AS intron #2 were transfected in CHO-S and HEK293 cells. After 3-6 days the cells were analysed using flow cytometry. A reduction of the amount of Ys in the poly(Y) tract leads to a modest increase in the population of cells that are double positive for dsRED and GFP (see FIG. 3). The constructs expressing the highest relative rates of GFP were the constructs I4 (0Y), I4 (5Y-5) and I4 (SYnude) containing significantly less Y in the poly(Y) tract (between 0 and 5) compared to the unchanged cTNT-I4 (27 Ys). This seems to confirm that a decrease in the strength of the splice acceptor in position AS intron #2 leads towards exclusion of GS exon #3 (coding for dsRED) and therefore higher expression from GS exon #4 (coding for GFP).

From the expression of these early constructs, it was clear that the basal expression level of the new construct was much in favour of dsRED expression. It has been described for the chicken troponin alternate exon that the size of the exon is a key factor of the alternative splicing event. Xu et al., 1993 (Mol Cell Biol, 13(6): 3660-74) describe that artificial exons smaller than 49 nucleotides are not recognized by the splice machinery if they lack a splice enhancer element (which is not present in the construct of the invention). On the other hand they show that exons with a size between 49 and 119 nucleotides are alternatively spliced. The exon with dsRED has a size of 718 nucleotides (6 times the maximum exon size analysed by Xu et al., supra) and is mainly included. Therefore the shift towards expression of the first exon might be simply due to the size of the exon.

The changes in shift in expression from dsRED to GFP by modifications in the poly(Y) were disappointing compared to data described in the literature (for example compared to the changes described in Fallot et al, 2009 (Nucleic Acids Res, 37(20):e134). Clearly alternate splicing could not be obtained by simply reducing the poly(Y) content of the intron upstream of the alternative exon.

The intron cTNT-I5, cloned downstream of the alternate exon (AS intron#3) has a rather reduced poly(Y) tract containing only 10 Ys. As the reduction of the number of Ys in AS intron #2 (which might lead to a weakening of the splice acceptor strength) favoured a shift towards GFP expression, it was speculated, that an increase in the content of Ys in AS intron#3 might lead to an increase in the splice acceptor strength and therefore to a shift from dsRED to GFP expression. Modified cTNT-I5 intron sequences containing up to 28 Ys (compared to the 10 that were present in the original construct) were cloned in position AS intron#3 (see FIG. 2b for an alignment of the sequences). Nevertheless no significant shift in GFP expression was observed (FIG. 3). Therefore the original cTNT-I5 sequence was used for analysis of the effect of modifications of the branch point and the intron-exon consensus region.

Transfection of Constructs with Modifications in the Branch Point and in the Intron-Exon Border

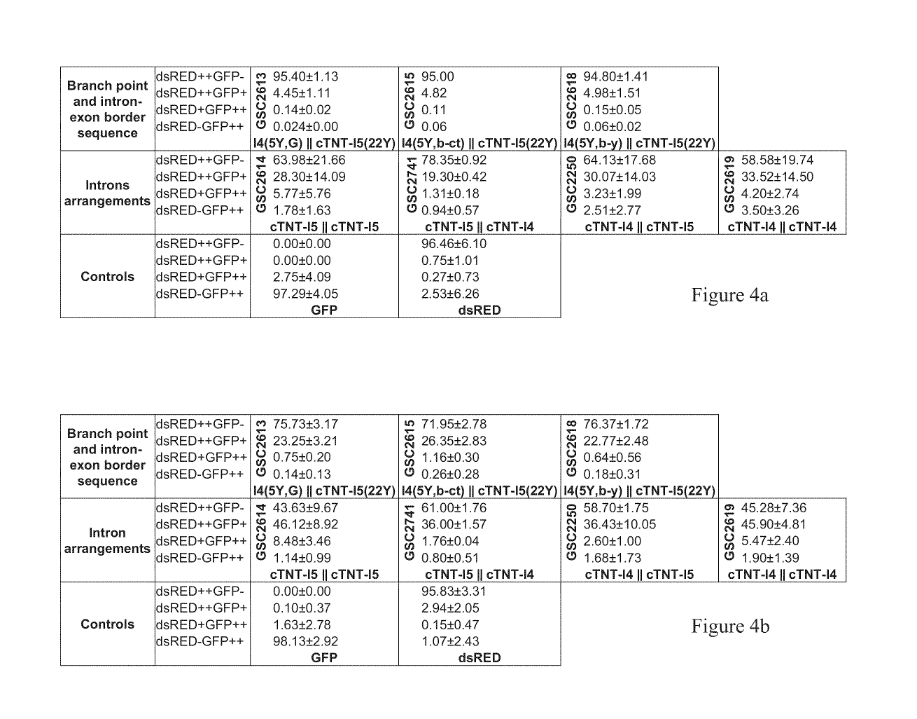

In order to further shift the splice ratio in favour of GFP expression, sequence modifications were introduced in the branch point region and in the intron-exon consensus region of AS intron #2, upstream of the alternate exon (exon #3 in FIG. 1a). These modifications were thought to further decrease the strength of the splice acceptor region. Details of the modifications introduced are shown in the alignment in FIG. 2b. None of these modifications led to a significant shift from dsRED to GFP expression (see FIG. 4, top row). This was surprising, as these modifications have been shown to have a huge impact on alternate splicing (see for example Fallot et al, supra).

Additionally, the introns cTNT-I4 and cTNT-I5 were rearranged in different ways. First, intron cTNT-I4 and cTNT-I5 were exchanged, so that the alternate exon expressing dsRED was flanked by cTNT-I5 in position AS intron #2 and by cTNT-I4 in position AS intron #3. Then, the sequence cTNT-I4 was used for AS intron#2 and AS intron #3. The same was done using the intron sequence cTNT-I5. Flanking the alternate exon with two identical introns increased the double positive (dsRED and GFP) population significantly. The best construct in HEK293 and CHO-S cells (GSC2614; cTNT-I5|cTNT-I5) increased the double positive population significantly (see FIG. 4, middle row). Construct GSC2619, having the orientation cTNT-I4|cTNT-I4 also showed a significant increase of the amount of double positive cells in CHO-S and HEK293 cells and was used for further constructs. This was highly surprising, as there is no literature suggesting that the similarity of introns flanking the alternative exon might have an impact on the splice ratio. Nevertheless our data suggest that two identical introns flanking an exon lead to alternative splicing of exons. This was shown for chicken troponin intron 4, chicken troponin intron 5 and also for the constitutively cut first intron of the human EF1alpha gene (shown in Example 3).

Combination of Poly(Y) and Branch Point Modifications in the cTNT-I4|cTNT-I4 Combination

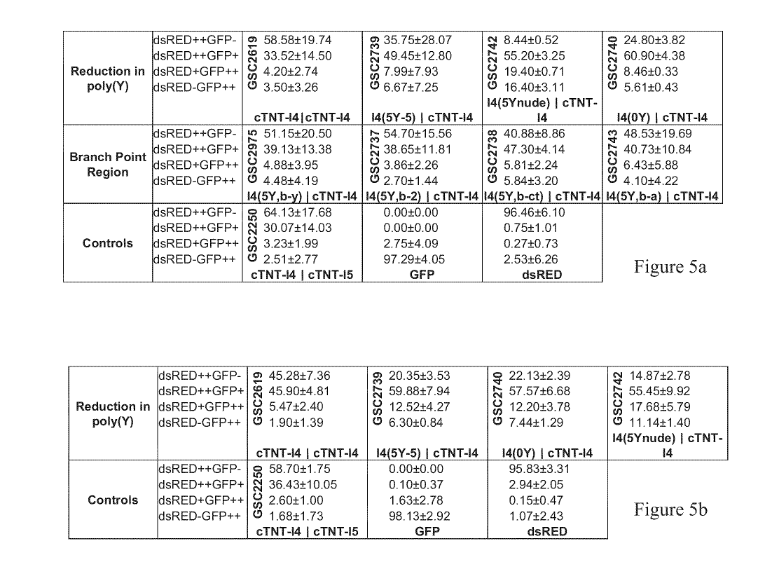

In the previous experiments a significant, but minor shift towards the GFP could be observed for constructs with reduced content of Y in the poly(Y) tract and of constructs having the same intron flanking the alternate exon (orientation cTNT-I4|cTNT-I4 or cTNT-I5|cTNT-I5). In order to analyse whether combining these modifications would lead to a further shift towards the expression of GFP, modifications of the poly(Y) tract and the branch point of AS intron#2 were introduced in the construct GSC2619 containing the cTNT-I4 intron up- and downstream of the alternate exon (orientation cTNT-I4|cTNT-I4). For these experiments the poly(Y) modifications showing the highest shift towards GFP expression were used (I4(5Y-5), I4(0Y), I4(5Ynude)). The construct GSC2250 (cTNT-I4|cTNT-I5) was included as a reference for the splice ratio of the basal construct. The combination of poly(Y) tract reduction and the use of cTNT-I4|cTNT-I4 configuration showed a significant shift towards GFP expression for all three constructs in HEK293 and CHO-S cells (FIG. 5a middle row and FIG. 5b top row). Interestingly, the combination of the use of the same intron (here cTNT-I4) and the combined reduction of the poly(Y) tract had a synergistic effect on the shift of the splice ratio towards the second open reading frame. On the other hand, the combination of modifications in the branch point regions and the reduction of the poly(Y) tract using the I4(5Y)|cTNT-I4 construct did not lead to a significant shift from dsRED to GFP (see FIG. 5a top row).

### Elimination of the Splice Donor Site

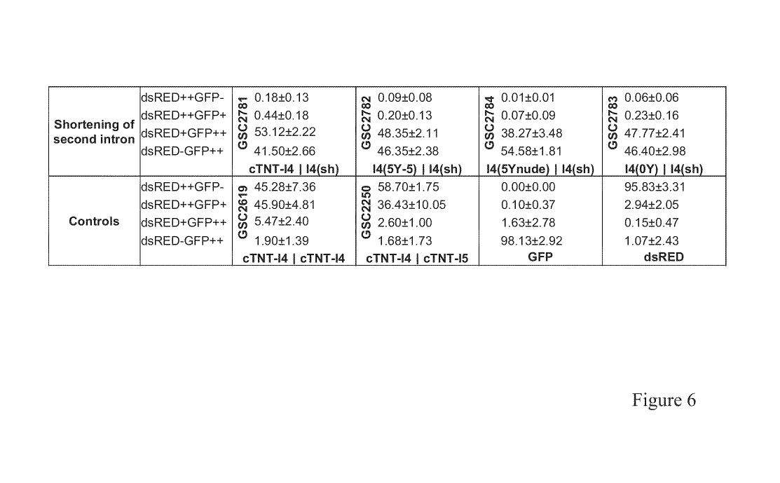

In order to shift the splice ratio from the first exon expressing dsRED to the second exon expressing GFP even further, the splice donor site of cTNT-I4 in position AS intron #3 was eliminated (see FIG. 2c for alignment). This was done by deleting the exon-intron consensus region and the entire intron upstream (5′) of the splice acceptor region (branch point, poly(Y) and intron-exon consensus were not modified) of AS intron #3. The elimination of the splice donor further increased the shift from dsRED expression to GFP expression. In combination with the reduction of Ys in the poly(Y) tract, this led to almost predominant GFP expression (FIG. 6).

### Summary on GFP-dsRED Expression Experiments

Different designs of alternate splicing constructs were tested based on the cTNT alternate exon 5 flanking introns. The basic construct (cTNT-I4|cTNT-I5) showed a preference for inclusion of the alternate exon and expressed mainly dsRED, the reporter protein expressed on the first open reading frame. It has been shown in literature that the size of the alternate exon has a major impact on the exclusion (in case of small exons) or inclusion (in case of larger exons) of the alternative exon. The reduction of the amount of Ys in the poly(Y) tract and the use of the same intron up- and downstream of the alternate exon, in particular the cTNT-I4 was shown to lead to a significant shift from dsRED expression (on the alternate exon) towards the expression of GFP (expressed on the second open reading frame). This shift could be further increased by combining the poly(Y) reduction and the cTNT-I4 up- and downstream of the alternate exon. This was a surprising finding, as the current literature does not suggest that the use of the same intron sequence up- and downstream of an exon leads to a shift towards exclusion of the flanked exon. Even more surprising, this effect could be confirmed using the EF1alpha first intron. This intron usually is not subject to alternative splicing. This demonstrates a general mechanism leading to alternative splicing.

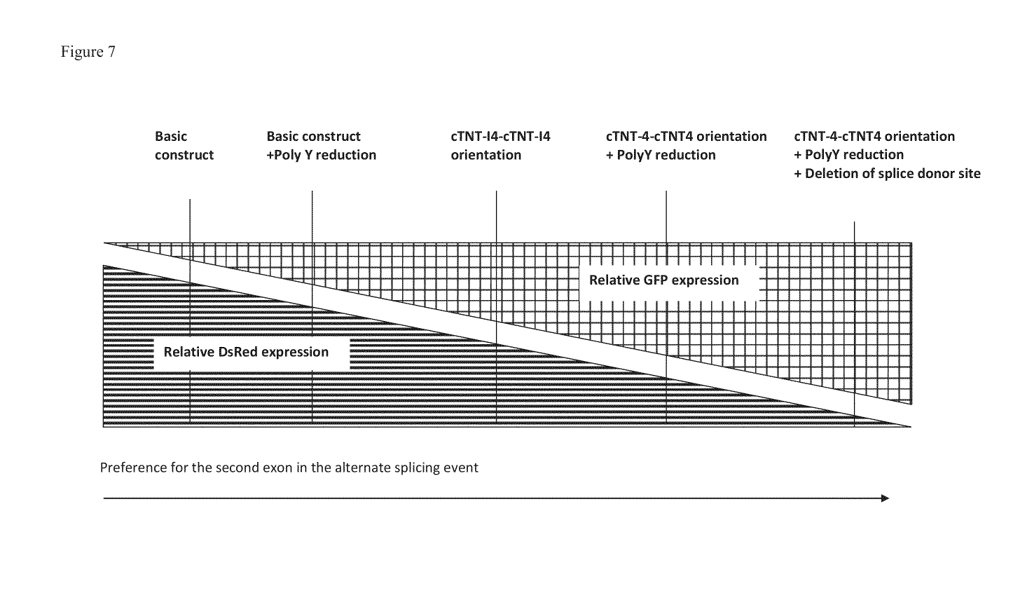

Finally, the deletion of the splice donor site downstream of the alternate exon (AS intron #3) led to further exclusion of the alternate exon. The cells transfected with these constructs seemed to express mainly GFP. The final alternate splicing constructs covered both extremes of alternate splicing (mainly inclusion of the alternate exon leading to predominant dsRED expression to mainly exclusion of the alternate exon leading to predominant GFP expression) as well as intermediate ratios (see FIG. 7 for a schematic drawing).

As mentioned above, it cannot be totally excluded that the fluorescence signal per protein, the detection level and the production efficiency of the two reporter proteins used are significantly different. Nevertheless, the three conditions identified above (usage of same intron before and after alternate exon, decrease the amount of Ys in the poly(Y) tract, elimination of the splice donor site) should be also valid for different proteins expressed using alternate splicing.

TABLE 5
List of Constructs

| Name of plasmid | SEQ ID No. |
|---|---|
| GSC 2250 | 38 |
| GSC 2329 | 39 |
| GSC 2330 | 40 |
| GSC 2323 | 41 |
| GSC 2619 | 42 |
| GSC 2781 | 43 |
| GSC 2342 | 44 |
| GSC 2328 | 45 |
| GSC 2321 | 46 |
| GSC 2324 | 47 |
| GSC 2339 | 48 |
| GSC 2334 | 49 |
| GSC 2336 | 50 |
| GSC 2340 | 51 |
| GSC 2331 | 52 |
| GSC 2453 | 53 |
| GSC 2325 | 54 |
| GSC 2332 | 55 |
| GSC 2341 | 56 |
| GSC 2326 | 57 |
| GSC 2454 | 58 |
| GSC 2327 | 59 |
| GSC 2338 | 60 |
| GSC 2335 | 61 |
| GSC 2333 | 62 |
| GSC 2337 | 63 |
| GSC 2322 | 64 |
| GSC 2617 | 65 |
| GSC 2739 | 66 |
| GSC 2782 | 67 |
| GSC 2621 | 68 |
| GSC 2740 | 69 |
| GSC 2783 | 70 |
| GSC 2622 | 71 |
| GSC 2742 | 72 |
| GSC 2784 | 73 |
| GSC 2620 | 74 |
| GSC 2737 | 75 |
| GSC 2615 | 76 |
| GSC 2743 | 77 |
| GSC 2738 | 78 |
| GSC 2618 | 79 |
| GSC 2975 | 80 |
| GSC 2613 | 81 |
| GSC 2614 | 82 |
| GSC 2741 | 83 |
| GSC 2780 | 84 |

### Example 2

### Stable Cells Expressing dsRED and GFP

### Materials and Methods

Materials and Methods for Example 2 were the same as those described for Example 1.

### Results

### Cloning of the Expression Construct

Different constructs for alternate splicing of a pre-mRNA leading to expression of GFP and dsRED have been described in Example 1. One of the constructs was chosen for development of a stable CHO cell line. As the pGLEX3 vector backbone is best suited for transient expression in HEK293 cells, the alternate splicing cassette of the selected construct GSC 2739 was inserted in the proprietary expression vector pGLEX41 (batch number GSC281). In this vector the alternate splicing cassette is driven by the mCMV promoter, which is well suited for stable expression in CHO cells. The expression cassette was cut out using the enzymes NheI and BstBI and cloned into the backbone of pGLEX41 opened using the same enzymes and CIPed. The resulting vector was called pGLEX41-ASC-cTNT-I4(5Y-5)|cTNT-I4-dsRED-GFP and received the batch number GSC3166 (SEQ ID NO: 111). The vector conferring the resistance genes against the antibiotic puromycin was pSEL3, a pGL3 (Promega, Madison, Wis.) derived vector. The puromycin resistance in this vector is under control of the SV40 promoter.

### Stable Transfection

The routine cell culture and the transfection of CHO-S have been described in Example 1. The DNA cocktail used for this transfection leading to stable cell lines was a mix of 95% pGLEX41 and 5% of pSEL3 (molar ratio). After the transfection, the cells were incubated for one day on an orbital shaker. The following day, the cells were plated in different dilutions on 96 well plates under selection pressure. The concentration of puromycin used for selection reliably yields stable populations that are referred to as “minipools”, because they can be a mix of different stable integration events, rather than clonal populations. After one week the selection pressure was refreshed. Screening for wells containing minipools was performed after two weeks using an Elisaplate reader. Cells showing high fluorescence signal were expanded to 24 well plate scale and analysed by FACS. In order to obtain clonal populations, one minipool was chosen for a second round of limiting dilution. For this the cells were diluted at different concentrations and plated in 96 well plates. Clonal populations were selected and expanded based on the amount of colonies growing on a plate and the absence of multiple growth centres in a well. After expansion to 24 well, the dsRED and GFP expression of the clonal populations were assessed by FACS.

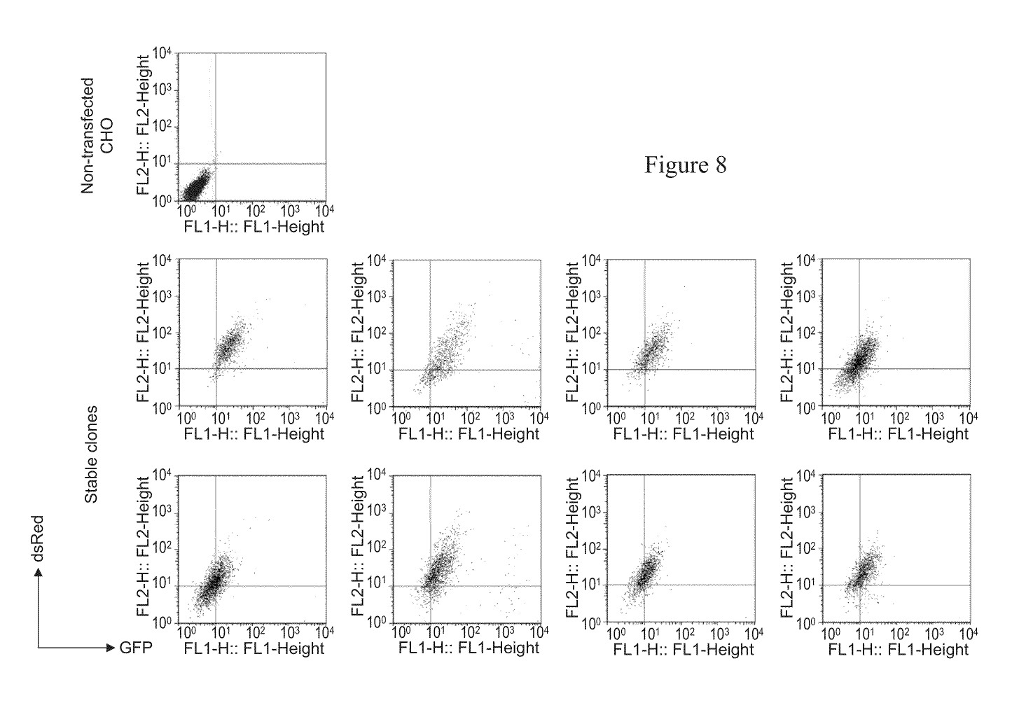

A comparison of the relative expression levels of dsRED and GFP of the clones obtained after limiting dilution 2 showed a very similar ratio of dsRED to GFP expression for most clones, although the overall expression level varies between different clones. All clones were double-positive for dsRED and GFP. No clone was observed that expressed only GFP or dsRED. FIG. 8 shows exemplary GFP and dsRED expression of 8 randomly chosen clones.

The similar splicing ratio of different clones derived from the same parental minipool shows that the splice ratio remains stable over multiple generations, without shifts towards one of the two exons. This indicates that the alternate splicing ratio is mostly defined by the DNA construct, although every clone might have a slightly different splicing ratio for the alternate exons (leading to minor differences in the ratio of GFP to dsRED expression). It also indicates that there is no strong selection pressure against the use of alternate splicing for expression of recombinant proteins, otherwise many clones would have lost expression.

In summary, clonal populations generated in this example show that the alternate splicing construct of the invention allows stable expression at an unchanged ratio for multiple generations without the use of selection pressure.

### Example 3

### Transient Expression of Antibodies

### Materials and Methods

### Cloning of Constructs

An anti-HER2 antibody was used in the preparation of a reporter construct. Heavy and light chains of the anti-HER2 antibody were codon-optimized for expression in CHO cells. The genes were cloned in both possible combinations in the position of GFP and dsRED of the vectors described in Example 1. Selected constructs were cloned in the plasmid pGLEX41 for further analysis. In this vector the expression of the alternate splicing construct is controlled by the mouse CMV promoter.

### Transfection of Cells and Quantification of Secreted Anti-HER2 Antibody

The constructs were transfected in CHO-S cells and HEK293 cells in 24 well format or 50 ml bioreactor format as described in Examples 1 and 2. After transfection the cells were incubated on a shaken platform at 37° C., 5% CO2 and 80% humidity. The secreted antibody was quantified 3 to 6 days after transfection using the Octet QK system (Fortebio) with Protein A bioprobes according to the specifications of the manufacturer. The calibration curve was done using the purified anti-HER2 antibody.

### Transient Expression of Anti-HER2 Using Alternate Splicing Constructs

The anti-HER2 antibody was used as a model protein for the expression of antibodies using alternate splicing. This antibody is well expressed and stable in culture supernatants during the production phase. It was shown in previous co-transfection experiments that this anti-HER2 antibody is better expressed if the heavy chain is transfected in a two-fold molar excess over the light chain. This ratio was shown to depend on the respective antibody. Therefore the best constructs in this study might show high expression only for the anti-HER2 antibody in question. Other antibodies might have a different optimal ratio of heavy to light chain and might require different splicing constructs.

The open reading frames coding for the anti-HER2 antibody heavy and light chains were cloned in two different orientations (orientation 1: first light chain, then heavy chain; orientation 2: first heavy chain, then light chain) in the position of the two fluorescence markers GFP and dsRED of Example 1.

As described in Example 1, the first intron (AS intron #1) is a constitutively spliced intron sequence that is present in all constructs. The second intron (AS intron #2) is located upstream of the alternate exon, which contains the first of the two open reading frames. The third intron (AS intron #3) is downstream of the alternate exon. This intron is upstream of the exon containing the second open reading frame. Depending on the splice event the final mature mRNA will code either for the open reading frame 1 on the alternate exon or for open reading frame 2 (see FIG. 1a for a schematic drawing of the alternate splicing events).

Expression constructs with varying amount of poly(Y) were selected from the preliminary study using GFP and dsRED (see Table 1) based on the absolute expression level and the shift in the expression from the first (dsRED) to the second open reading frame (GFP). These were combined with the full length AS intron #3 or the shortened version (“sh”) that was shown to lead to efficient expression of the second open reading frame.

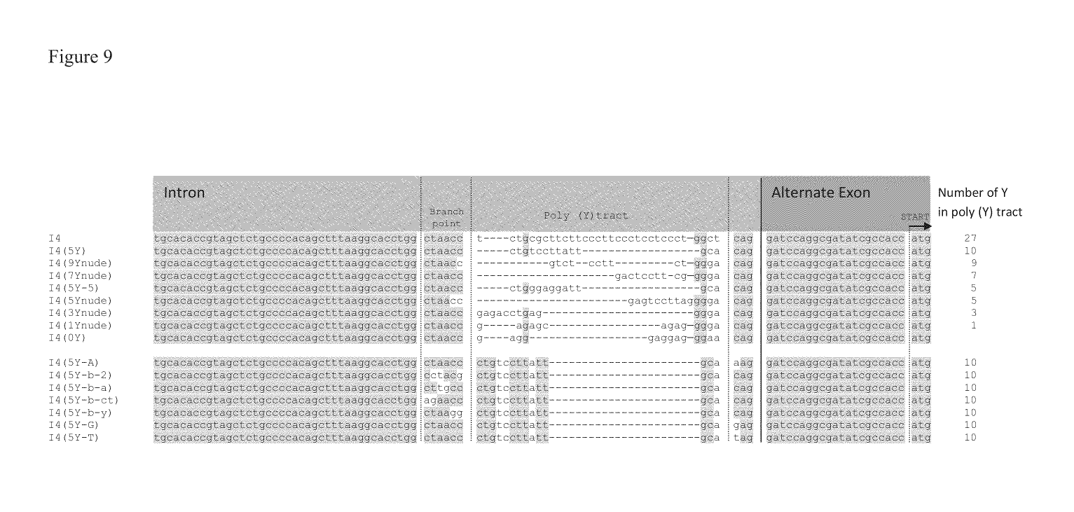

In order to check whether constructs showing only a minor shift in the dsRED to GFP ratio could have an influence of the expression level of the anti-HER2 antibody, some of the constructs that were showing no obvious effect (branch point modifications and the intron-exon consensus region modifications) were reassessed using the anti-HER2 antibody as reporter protein and the influence of the poly(Y) tract was analysed more in detail (see Table 6 for all constructs and the alignments in FIG. 9 for sequence information).

For expression of an antibody, both heavy and light chain have to be expressed at relevant levels, and it was shown that for the anti-HER2 antibody, a two-fold excess of HC expression is favourable for the antibody secretion in transient transfections. Constructs with a different amount of Y in the poly(Y) tract were cloned and transfected in CHO-S cells. On day six the amount of accumulated anti-HER2 antibody in the supernatant was quantified by Octet.

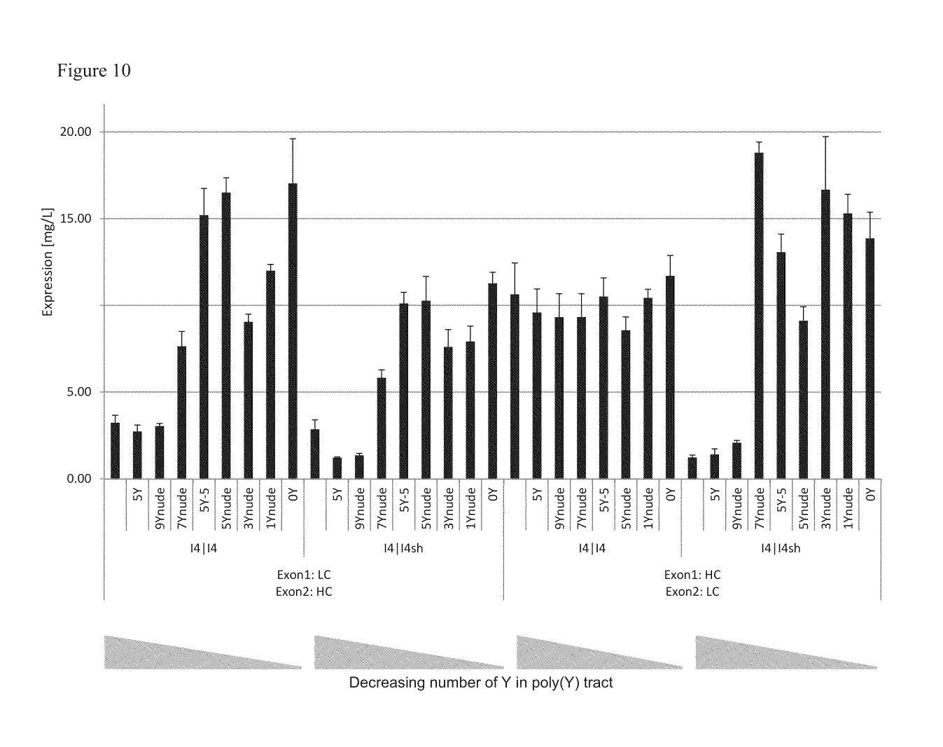

The expression levels of constructs with orientation LC-HC and orientation HC-LC are shown in FIG. 10. The overall expression level is highest in orientation LC-HC, with the light chain on the alternate (first) exon and a full length second intron. The titers obtained were up to 60% of the co-transfection control using the optimal ratio of heavy to light chain. This shows the potential of alternate splicing for the expression of antibodies.

The expression level of all constructs increased with a decreasing amount of Ys in the poly(Y) tract (with the exception of the series I4I4 in orientation HC-LC). Less Ys in the first intron shift the splicing ratio away from the predominantly expressed first exon to the second alternate exon and hence to higher relative expression of the open reading frame present on the second alternate exon. As the antibody needs expression of heavy and light chain for successful assembly and secretion, this is beneficial to the expression of the entire antibody. It was observed, that the expression level starts to increase significantly if the poly(Y) tract has 7 or less Ys. This might be when the alternate splicing is shifted towards approximately equimolar expression of the two alternate exons (because the effect is observed for the I4I4sh constructs in both orientations). Surprisingly, the shortening of AS intron #3 has little effect on the amount of Ys in the poly(Y) tract leading to best expression. This might be due to the insensitivity of the reporter system, allowing a relatively wide range of the HC:LC ratio.

TABLE 6
List of constructs based on pGLEX3 made for anti-HER2 antibody expression. SEQ ID Nos:
85 to 102 comprise the first exon of the mRNA up to the start codon (ATG) of the first
open reading frame. SEQ IDs 103 to 108 start with the stop codon of the first open reading
frame and terminate with the start codon of the second open reading frame.

| | | LC-HC | | | HC-LC | | |
|---|---|---|---|---|---|---|---|
| | | cTNT-I4 | cTNT-I5 | I4(sh) | cTNT-I4 | cTNT-I5 | I4(sh) |
| | Ys in | SEQ ID | SEQ ID | SEQ ID | SEQ ID | SEQ ID | SEQ ID |
| | construct | No: 103 | No: 104 | No: 105 | No: 106 | No: 107 | No: 108 |
| Ys in construct | — | | | | | | |
| cTNT-I4 | 27 | GSC2821 | GSC2822 | GSC3164 | GSC2816 | GSC2819 | GSC3170 |
| I4 (5Y) | 10 | GSC4218 | | GSC4228 | GSC4222 | | GSC4225 |
| SEQ ID No: 085 | | | | | | | |
| I4 (9Ynude) | 9 | GSC4344 | | GSC4339 | GSC4335 | | GSC4336 |
| SEQ ID No: 086 | | | | | | | |
| I4 (7Ynude) | 7 | GSC4345 | | GSC4355 | GSC4337 | | GSC4341 |
| SEQ ID No: 087 | | | | | | | |
| I4 (5Y − 5) | 5 | GSC2820 | | GSC4226 | GSC4217 | | GSC4221 |
| SEQ ID No: 088 | | | | | | | |
| I4 (5Ynude) | 5 | GSC4220 | | GSC4215 | GSC2823 | | GSC4223 |
| SEQ ID No: 089 | | | | | | | |
| I4 (3Ynude) | 3 | GSC4340 | | GSC4354 | | | GSC4333 |
| SEQ ID No: 090 | | | | | | | |
| I4 (1Ynude) | 1 | GSC4332 | | GSC4407 | GSC4331 | | GSC4405 |
| SEQ ID No: 091 | | | | | | | |
| I4 (0Y) | 0 | GSC2818 | | GSC4224 | GSC3151 | | GSC4214 |
| SEQ ID No: 092 | | | | | | | |
| I4(5Y, b − ct) | | GSC2977 | | | GSC3154 | | |
| SEQ: ID No: 093 | | | | | | | |
| I4 (5Y, b − y) | | GSC3182 | | | | | |
| SEQ ID No: 094 | | | | | | | |
| I4(5Y, b − 2) | | GSC2985 | | GSC3155 | GSC2984 | | GSC3147 |
| SEQ ID No: 095 | | | | | | | |
| I4(5Y, b − a) | | GSC2986 | | | | | |
| SEQ ID No: 096 | | | | | | | |
| I4(5Y − A) | | GSC2976 | | | GSC3158 | | |
| SEQ ID No: 097 | | | | | | | |
| I4(5Y − 5, G) | | GSC3085 | | | | | |
| SEQ ID No: 098 | | | | | | | |
| I4 (5Ynude, A) | | GSC3089 | | | | | |
| SEQ ID No: 099 | | | | | | | |
| I4(5Ynude, b − 2) | | GSC3184 | | | | | |
| SEQ ID No: 100 | | | | | | | |
| I4(5Ynude, A) | | | | | GSC3153 | | |
| SEQ ID No: 101 | | | | | | | |
| I4(5Y − 5, G) | | | | | GSC3160 | | |
| SEQ ID No: 102 | | | | | | | |

For the constructs in the orientation LC-HC, the constructs 3Ynude and 1Ynude show less expression compared to constructs with less (0Y) or more Ys (5Ynude) in the poly(Y) tract. This shows that minor variations in the sequence also impact the splice ratio and that the number of Ys in the poly(Y) tract and the exon size are not the only factors influencing the splice efficiency.

In contrast to this, the I4I4-constructs with HC-LC orientation show a relative high expression level independent of the poly(Y) content. It has been described in the literature that increasing the length of the alternate exon shifts the splice ratio towards the alternate (first) exon (and therefore open reading frame 1). Using the shortened AS intron #3, the poly(Y) content influences the expression of the anti-HER2 antibody tested, and therefore the splice ratio. One explanation of these experimental results is that the large exon coding for the open reading frame of the heavy chain in the first position weakens the impact of the poly(Y) tract on the splice ratio, leading to a fixed ratio of the two splice variants. Only when the splicing event is further destabilized by shortening the second intron and the elimination of the splice donor of the second intron, the poly(Y) tract might influence the splice ratio.

In the screening described above, the constructs 5Y-5, 5Ynude and 0Y were identified as constructs giving the highest transient expression results for the orientation LC-HC. These expression constructs were cloned into the expression vector used for stable cell line development. As the pre-splicing RNA construct remains unchanged (only the promoter was changed) this cloning step was not expected to lead to significant differences in the splicing ratio.

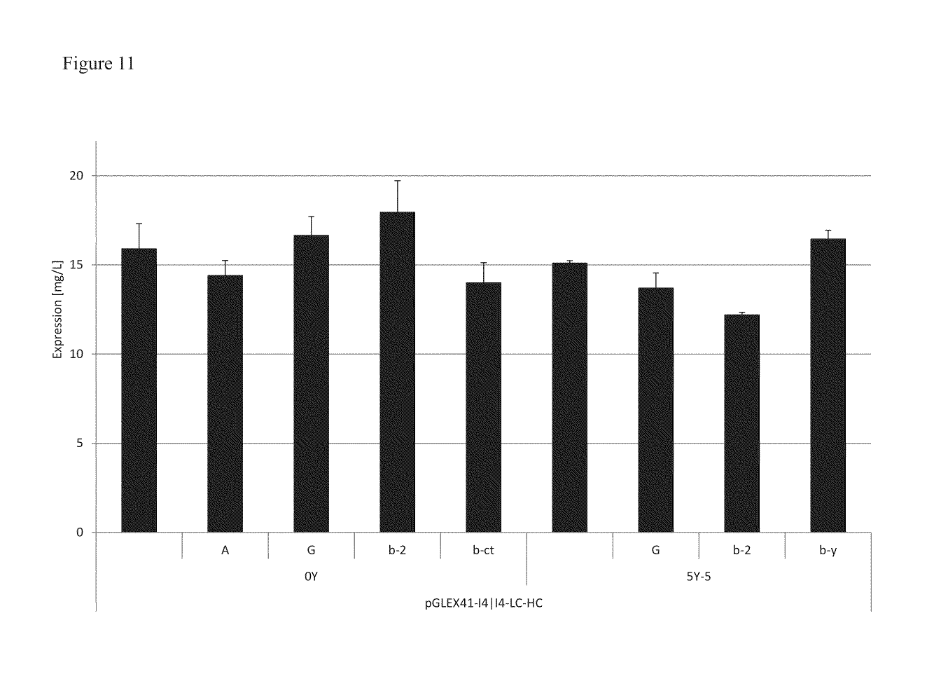

Using GFP and dsRED as reporter proteins, no effect of intron-exon consensus modifications or of branch point modifications could be observed (see Example 1). However, minor shifts in the splicing ratio might not be detectable using the GFP/dsRED reporter system. In order to verify whether intron-exon modifications or branch point modifications might be useful for fine tuning the splice ratio for antibody expression, new constructs were cloned based on the 5Y-5, 5Ynude and 0Y constructs in pGLEX41 (see Table 7 for complete list of constructs and FIG. 11 for expression results of the 0Y construct).

TABLE 7
List of constructs used for fine tuning of heavy chain to light chain
expression in the final vector pGLEX41. SEQ ID Nos: 88, 89, 92,
99, 100, 102 and 112 to 128 listed below, comprise the first exon
of the mRNA up to the start codon (ATG) of the first open reading
frame. SEQ ID No: 103 starts with the stop codon of the first open
reading frame and terminates with the start codon of the second
open reading frame

| | |
|---|---|
| | Intron constructs |
| | used downstream |
| | of the alternate exon |
| | (position AS intron #3) |
| Intron constructs used | cTNT-I4 (SEQ ID No: 103) |
| upstream of the alternate | GS number |

| exon (position AS intron #2) | LC-HC | HC-LC |
|---|---|---|
| I4 (0Y) | GSC3157 | GSC3151 |
| SEQ ID No: 92 | | GSC4219 |
| I4 (0Y, b-a) | GSC3436 | GSC3466 |
| SEQ ID No: 112 | | |
| I4 (0Y, b-ct) | GSC3432 | GSC3470 |
| SEQ ID No: 113 | | |
| I4 (0Y, b-y) | GSC3439 | GSC3465 |
| SEQ ID No: 114 | | |
| I4 (0Y, b-2) | GSC3462 | GSC3465 |
| SEQ ID No: 115 | | |
| I4 (0Y, A) | GSC3447 | GSC3442 |
| SEQ ID No: 116 | | |
| I4 (0Y, T) | GSC3453 | GSC3430 |
| SEQ ID No: 117 | | |
| I4 (0Y, G) | GSC3434 | GSC3446 |
| SEQ ID No: 118 | | |
| I4 (5Ynude) | GSC3162 | GSC3169 |
| SEQ ID No: 89 | | |
| I4 (5Ynude, b-a) | GSC3460 | GSC3441 |
| SEQ ID No: 119 | | GSC3449 |
| I4 (5Ynude, b-ct) | | GSC3461 |
| SEQ ID No: 120 | | |
| I4 (5Ynude, b-y) | GSC3451 | GSC3444 |
| SEQ ID No: 121 | | |
| I4 (5Ynude, b-2) | GSC3464 | GSC3433 |
| SEQ ID No: 100 | | |
| I4 (5Ynude, A) | GSC3448 | GSC3458 |
| SEQ ID No: 99 | | |
| I4 (5Ynude, T) | GSC3457 | GSC3450 |
| SEQ ID No: 122 | | |
| I4 (5Y-5) | GSC-3150 | |
| SEQ ID No: 88 | | |
| I4 (5Y-5, b-a) | GSC3455 | GSC3463 |
| SEQ ID No: 123 | | |
| I4 (5Y-5, b-ct) | GSC3431 | |
| SEQ ID No: 124 | | |
| I4 (5Y-5, b-y) | GSC3467 | GSC3429 |
| SEQ ID No: 125 | | |
| I4 (5Y-5, b-2) | GSC3454 | |
| SEQ ID No: 126 | | |
| I4 (5Y-5, A) | GSC3456 | |
| SEQ ID No: 127 | | |
| I4 (5Y-5, T) | | GSC3459 |
| SEQ ID No: 128 | | GSC3468 |
| I4 (5Y-5, G) | GSC3452 | GSC3437 |
| SEQ ID No: 102 | | |

As shown in FIG. 11, neither the branch point modifications nor the intron-exon consensus region showed a significant increase in the anti-HER2 antibody titers obtained in transient transfection. These modifications seem to be neutral (ATG) or negative (for example b-y) for the expression.

As only minor differences were observed in the expression level of branch point and intron-exon modifications, the two constructs for stable cell line development were chosen on convenience and availability. Both constructs show similar expression levels: I4(0Y)-I4 and I4(0Y, b-2)-I4.

### Alternate Splicing is Enhanced if the Alternate Exon is Flanked by Similar Introns

In previous experiments (Example 1) it was observed that using the same intron (either the cTNT intron #4 or the cTNT intron #5) up- and downstream of the alternate exon leads to higher expression of the second open reading frame. In order to analyse whether this is only true for introns naturally involved in alternate splicing, a constitutive intron from the human EF1alpha gene was used for the expression of an anti-HER2 antibody. The EF1alpha intron was cloned up- and downstream of the alternate exon. Intermediate constructs with EF1alpha as first intron and cTNT-I4 as second intron were cloned as well.

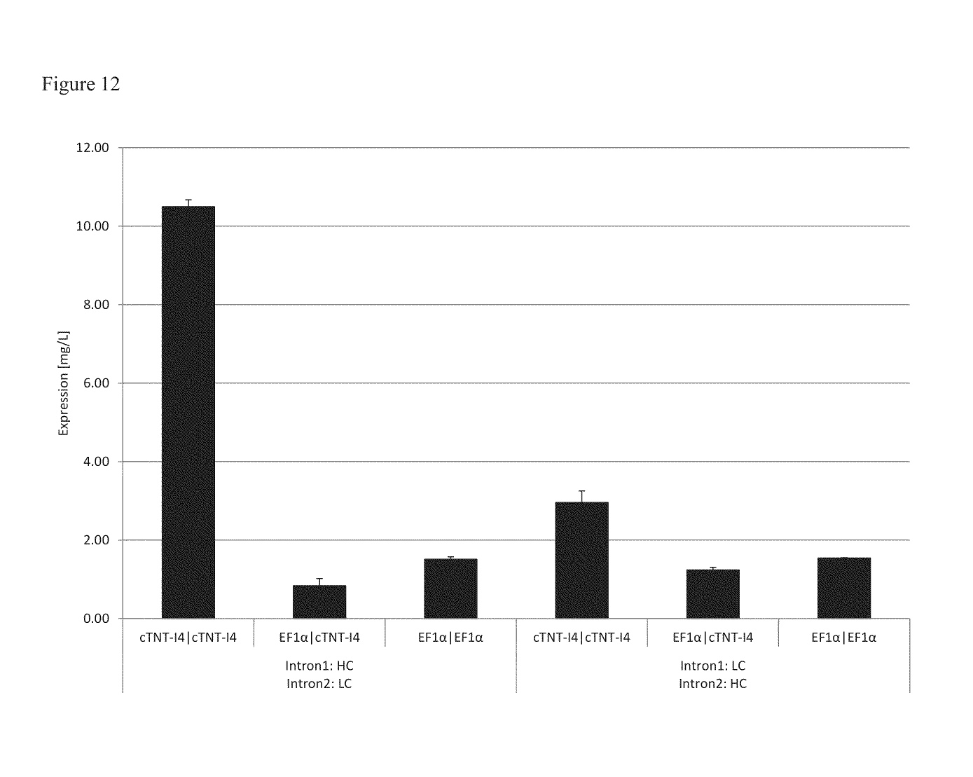

The results are shown in FIG. 12. Constructs with identical introns flanking the alternate exon up- and downstream show higher expression levels compared with constructs having different introns, independent of whether the heavy or light chains of the anti-HER2 antibody are expressed on the alternate exon.

Using the cTNT introns the expression level is higher compared to the EF1alpha introns, although the human EF1alpha intron was described to have an enhancer activity. This surprising result shows that using introns involved naturally in alternate splicing leads to higher expression of the second exon and hence to better expression of multimeric proteins like antibodies. Another example of using the same intron flanking the alternate exon was shown with the cTNT-Intron 5 in Example 1. Here as well the use of the same intron lead to a more equilibrated expression of the two alternate exons.

### Example 4

### Creation of Stable Cell Lines Expressing Anti-HER2 Antibody

In order to obtain stable expression of the reporter anti-HER2 antibody in CHO-S cells, the alternate splicing construct I4(0Y)I4-anti-HER2-LC-HC described in Example 3 was cloned in the expression vector pGLEX41 under control of the mouse CMV promoter and the Ig variable region intron and splice acceptor sequence (Bothwell et al., supra). This cloning step leads to the vector pGLEX41-ASC-I4(0Y)I4-anti-HER2-LC-HC.

Two additional vectors carry the resistance genes for puromycin and neomycin. Both resistance genes are under control of the SV40 promoter.

The cells were transfected using JetPEI™ (Polyplus-transfections, Strasbourg, France) following the procedure recommended by the manufacturer. The expression vector carrying the product gene and the two vectors providing the genes for resistance to the antibiotics used for selection (puromycin and geneticin) were linearised and co-transfected into the CHO-S (cGMP banked) host cells. The plasmids are introduced at a random integration site in the genome of the CHO-S host cell line. In our hands, this process is highly reproducible for rapidly and efficiently generating stable high expressing cell lines.

The transfection as well as the subsequent cultivation of the cells was performed in animal derived components free media. The day after the transfection, cells were seeded in selective medium (growth medium containing puromycin and geneticin) into 96 well plates at different cell densities. Both antibiotics are efficient inhibitors of protein biosynthesis. The high selection pressure due to the double selection efficiently eliminates not only untransfected cells but also non- and low-producer clones. After one week of incubation at 37° C., 5% CO₂, and 80% humidity, the selection pressure was renewed by addition of 1 volume of selective medium to the cells. After another week of static incubation the dilutions yielding less than 30% of wells showing growth were identified. The supernatants of the wells showing growth were analysed for accumulated anti-HER2 antibody using the Octet (Fortebio, Manlo Park, Calif.). The 72 minipools showing the highest expression were expanded first into 24 well plates, then into tubespin scale in suspension and assessed in a supplemented 14 days batch in tubespin 50 ml bioreactors. The highest titer obtained at the end of the batch culture was 197 μg/ml (see FIG. 13).

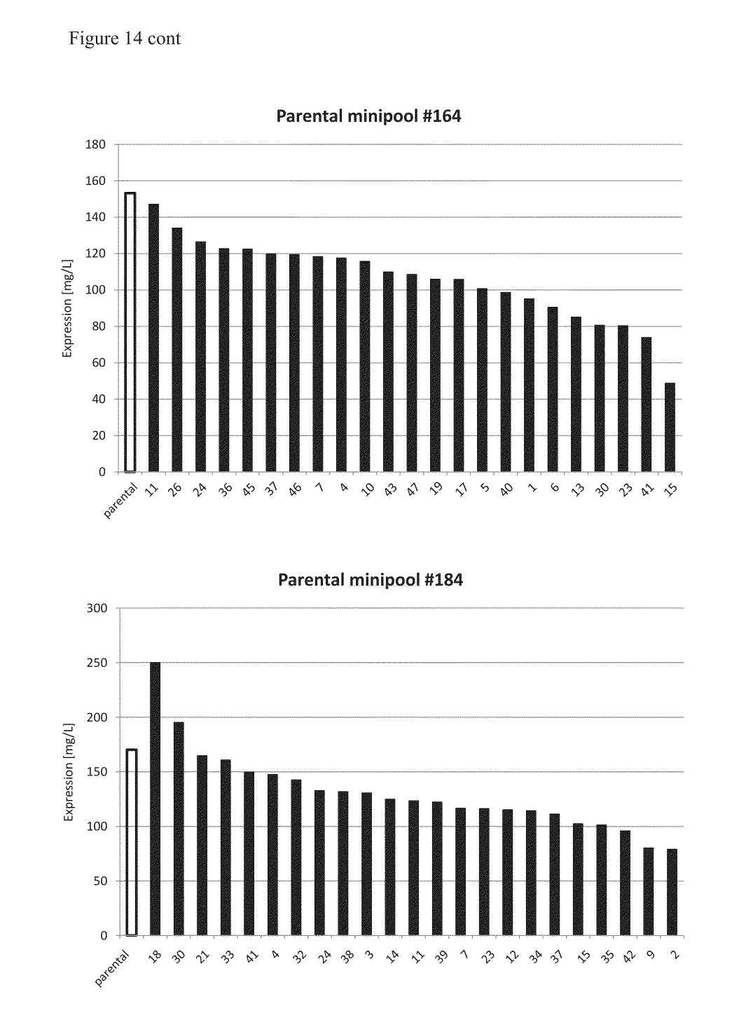

In order to obtain clonal populations, the four best expressing minipools with an expression level ranging from 150-197 μg/ml were chosen to undergo a second round of limiting dilution. This was done by plating the cells at different dilutions in growth medium in 96 well plates. After two weeks the number of colonies that had grown in the different dilutions was assessed. The clonal populations were expanded first to 24 well plate and then to 50 ml bioreactor tube scale. In this scale the highest titers obtained were 250 μg/ml in a supplemented non-optimized batch in 50 ml bioreactor tubes using 10 ml of medium (see FIG. 14). Compared to the usual titers obtained at this stage with the same antibody the maximum titer obtained with alternate splicing is around 3× lower. Nevertheless these titers represent the first industrially relevant production level of a stable cell line producing an antibody based on alternate splicing technology.

### Example 5

### Expression of Bispecific Antibodies Using Alternate Splicing Constructs

Bispecific antibodies are antibodies that have been engineered in order to recognize two different epitopes. A major problem in the development of bispecific antibodies for therapeutic applications is the production at an industrially relevant scale. Therefore the development of technologies that allow either higher expression of bispecific antibodies or production of the bispecific antibodies at higher purity (with lower contamination of the bispecific antibody by-products) are of upmost importance.

Bispecific antibodies are composed by multiple subunits. The number of subunits needed for expression depends on the chosen format. In an aspect of the present invention, bispecific antibody constructs are composed by three different subunits coding for a light chain, a heavy chain and an Fc-scFv. Similar to regular antibodies where the heavy chain and the light chain need to be transfected in an optimal ratio, bispecific constructs are best expressed at a specific ratio of the three subunits. This ratio depends on the bispecific antibody and also might vary from one format to another.

The alternate splicing expression cassettes developed in Examples 1-3 allow the simultaneous expression of two different proteins (GFP or dsRED) or subunits of the same protein (heavy chain and light chain of an antibody) at a fixed ratio. As it is favourable to express the subunits of the bispecific antibody at a certain molar ratio, the alternate splicing construct might prove useful for the expression of two subunits at the ratio leading to the highest expression or to the lowest contamination with by-products. An in-house generated bispecific antibody is composed of three different subunits: heavy chain, light chain and the Fc-scFv. For optimal expression of the correctly composed product, the ratio of heavy chain to Fc-scFv was shown to be the most important parameter in transient co-transfection experiments. The relative ratio of the light chain was of minor importance.

Based on this observation, the heavy chain and the Fc-scFv were cloned into the alternate splicing construct I4(7Y)I4sh described in Example 3, leading to the vectors GSC5642 (orientation: HC-scFv), GSC5643 (orientation: scFv-HC) and GSC5641 for the expression of the light chain.

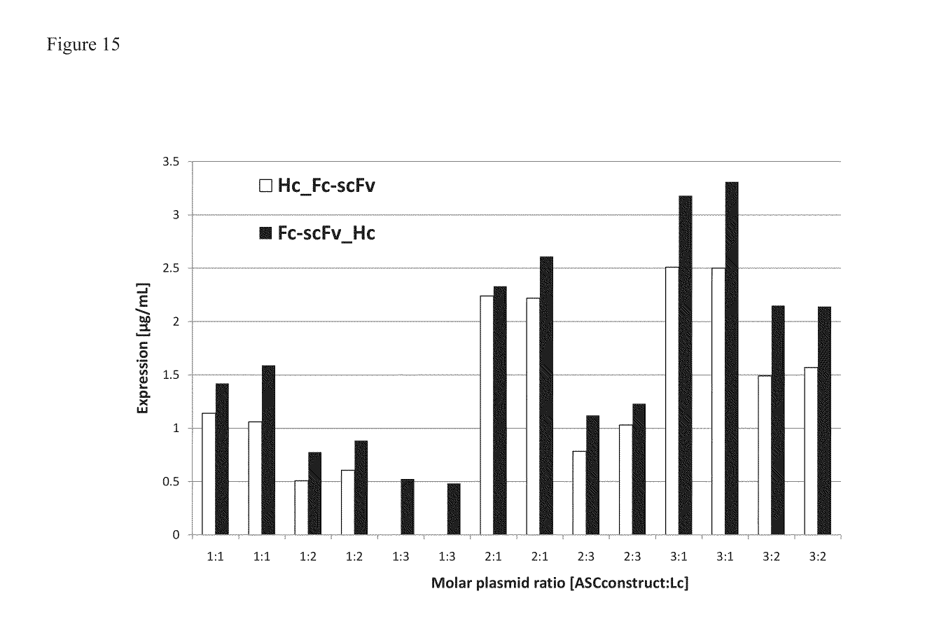

The vectors with the alternate splicing construct and the vector for the light chain were co-transfected in CHO-S cells using different ratios of the alternate splicing construct and the vector coding for the light chain. The expression levels of the resulting antibodies are shown in FIG. 15.

In general, the expression level increases for both constructs with increasing ratio of the alternate splicing construct over the light chain construct. Higher expression of light chain reduces the amount of antibody in the supernatant. The highest expression level was observed for a three-fold molar excess. As no plateau was observed, the true optimum might be an even higher molar excess. No experiment has been performed to optimize the expression level of bispecific antibodies or the level of by-products in the secreted proteins using varying amounts of poly(Y). Therefore there might be an additional potential for higher expression or lower by-product contamination in the used construct.

The presence of bispecific antibodies has been confirmed by ELISA (specific for the two arms of the bispecific antibody). The successful expression of bispecific antibodies using the alternate splicing construct I4(7Y)I4sh demonstrates that alternate splicing can be used for successful expression of regular antibodies as well as bispecific antibodies with more than two types of subunits. Expression at the optimal ratio might also be achieved by co-transfection (as it was done for identification of the optimal ratio). Nevertheless a major advantage of using the alternate splicing cassette is the possibility to directly translate the optimal ratio in a stable cell format.

1. An expression construct comprising in a 5′ to 3′ direction:
(i) a promoter;
(ii) an optional first splice donor site;
(iii) a first flanking intron;
(iv) a first splice acceptor site;
(v) a first exon encoding a first polypeptide;
(vi) an optional second splice donor site;
(vii) a second flanking intron;
(viii) a second splice acceptor site; and
(ix) a second exon encoding a second polypeptide,
(x) wherein upon entry into a host cell, transcription of the first exon results in expression of the first polypeptide and/or transcription of the second exon results in expression of the second polypeptide.

2. An expression construct according to claim 1, wherein the first and the second flanking introns are selected from the group consisting of: chicken troponin (cTNT) intron 4, cTNT intron 5 and first intron of the human EF1alpha gene.

3. An expression construct according to claim 1, wherein the first and the second flanking introns have a nucleic acid sequence homology of at least 80% for at least 50 nucleotides.

4. (canceled)

5. (canceled)

6. An expression construct according to claim 1, further comprising at least one polypyrimidine (poly(Y)) tract upstream or downstream of the first exon.

7. (canceled)

8. (canceled)

9. An expression construct according to claim 6, wherein the poly(Y) tract comprises less than 30 pyrimidine bases.

10. (canceled)

11. (canceled)

12. An expression construct according to claim 1, wherein the expression construct comprises a third splice donor site, an intron and a third splice acceptor site located downstream of said promoter.

13. An expression construct according to claim 12, wherein the splice donor site, intron and splice acceptor site are constitutive.

14. An expression construct according to claim 12, wherein the third splice donor site is preceded by a 5′UTR and/or the third splice acceptor site is followed by a 5′UTR.

15. An expression construct according to claim 1, wherein the flanking intron sequences are selected from the group consisting of: SEQ ID Nos: 129 to 175.

16. An expression construct according to claim 1, wherein the first polypeptide is an antibody heavy chain or fragment thereof and the second polypeptide is an antibody light chain or fragment thereof, or wherein the first polypeptide is an antibody light chain or fragment thereof and the second polypeptide is an antibody heavy chain or fragment thereof.

17. An expression construct according to claim 1, wherein the first polypeptide is an antibody heavy chain and the second polypeptide is a Fc-scFv or wherein the first polypeptide is a Fc-scFv and the second polypeptide is an antibody heavy chain.

18. A polynucleotide encoding an expression cassette according to claim 1.

19. A cloning or expression vector comprising one or more polynucleotides according to claim 18.

20. A host cell comprising one or more cloning or expression vectors according to claim 19.

21. (canceled)

22. (canceled)

23. (canceled)

24. A method of producing a polypeptide comprising culturing the host cell of claim 20 in a culture and isolating the polypeptide expressed from the culture.

25. A method of producing a bispecific antibody comprising culturing the host cell of claim 20 and isolating the polypeptide expressed from the culture.

26. A method of optimizing the expression level of a protein of interest encoded by one or more expression cassettes according to claim 1, comprising:
(i) using first and second flanking introns having a nucleic acid sequence homology of at least 80% for at least 50 nucleotides;
(ii) reducing the number of pyrimidine bases in a poly(Y) tract upstream of the first exon or increasing the number of pyrimidine bases in a poly(Y) tract downstream of the first exon; and/or
(iii) deleting a splice donor site upstream of the second flanking intron.

27. A method of optimizing the heterodimerization level of a protein of interest encoded by one or more expression cassettes according to claim 1, comprising:
(i) using first and second flanking introns having a nucleic acid sequence homology of at least 80% for at least 50 nucleotides;
(ii) reducing the number of pyrimidine bases in a poly(Y) tract upstream of the first exon or increasing the number of pyrimidine bases in a poly(Y) tract downstream of the first exon; and/or
(iii) deleting a splice donor site upstream of the second flanking intron.


SEQUENCE LISTING (175 sequences)

SEQ ID NO: 1 - DNA, 32 bp, Artificial
  Glnpr991_Primer
ggtcatttcgaatcattacttgtacagctcgt

SEQ ID NO: 2 - DNA, 24 bp, Artificial
  Glnpr1095_Primer
cgctggctagcgtttaaacttaag

SEQ ID NO: 3 - DNA, 48 bp, Artificial
  Glnpr1096_Primer
atcgttcgaatatgggccctctcgcacaccggtctcctcttcctcctc

SEQ ID NO: 4 - DNA, 29 bp, Artificial
  Glnpr1097_Primer
tatagggccctgtgagcaagggcgaggag

SEQ ID NO: 5 - DNA, 31 bp, Artificial
  Glnpr1098_Primer
gcgcttcgaatcattacttgtacagctcgtc

SEQ ID NO: 6 - DNA, 29 bp, Artificial
  Glnpr1099_Primer
tatagggccctctacaggaacaggtggtg

SEQ ID NO: 7 - DNA, 28 bp, Artificial
  Glnpr1100_Primer
attaaccggtgcctcctccgaggacgtc

SEQ ID NO: 8 - DNA, 51 bp, Artificial
  Glnpr1138_Primer
aattaagctagcgtttaaacttaagcttccttggattacaaggatgacgat

SEQ ID NO: 9 - DNA, 25 bp, Artificial
  Glnpr1139_Primer
gtggcgatatcgcctggatcctgag

SEQ ID NO: 10 - DNA, 38 bp, Artificial
  Glnpr1140_Primer
ccaggcgatatcgccaccatgggtgcctcctccgagga

SEQ ID NO: 11 - DNA, 43 bp, Artificial
  Glnpr1141_Primer
ctacctgaattcttccgttactacaggaacaggtggtggcggc

SEQ ID NO: 12 - DNA, 41 bp, Artificial
  Glnpr1142_Primer
gaggagaccggtgccaccatggagcaagggcgaggagctgt

SEQ ID NO: 13 - DNA, 63 bp, Artificial
  Glnpr1158_Primer
aattaagctagcgtttaaacttaagcttccttggaggacccagtacccggatctagaggtagg

SEQ ID NO: 14 - DNA, 40 bp, Artificial
  Glnpr1180_Primer
aattaaaccggtgccaccatggtgagcaagggcgaggagc

SEQ ID NO: 15 - DNA, 25 bp, Artificial
  Glnpr1181_Primer
gcgcggctagcgtttaaacttaagc

SEQ ID NO: 16 - DNA, 62 bp, Artificial
  Glnpr1182_Primer
ttgtgatatcgcctggatcctgtgcaataaggacagggttagccaggtgccttaaagctgtg

SEQ ID NO: 17 - DNA, 37 bp, Artificial
  Glnpr1183_Primer
agcaggatatcgcctggatcctgagacagggaggagg

SEQ ID NO: 18 - DNA, 62 bp, Artificial
  Glnpr1184_Primer
atatgatatcgcctggatcctgagccagggagcaggcaaggcaagaagcgcagaggttagcc

SEQ ID NO: 19 - DNA, 45 bp, Artificial
  Glnpr1185_Primer
agtcgatatcgcctggatcctgagccaggtagcagggaagggaag

SEQ ID NO: 20 - DNA, 62 bp, Artificial
  Glnpr1186_Primer
gatggatatcgcctggatcctgagccagggaggagggaaggcaacaagcgcagaggttagcc

SEQ ID NO: 21 - DNA, 25 bp, Artificial
  Glnpr1187_Primer
gcgcgaattcaggtagttactgcac

SEQ ID NO: 22 - DNA, 66 bp, Artificial
  Glnpr1189_Primer
tataaccggtctcctcttcctcctcgtcctcctgatcctcctgacctgagccagggaggagggaag

SEQ ID NO: 23 - DNA, 74 bp, Artificial
  Glnpr1190_Primer
taataccggtctcctcttcctcctcgtcctcctgatcctcctgacctgagccagggagcaggcaaggcaagaag

SEQ ID NO: 24 - DNA, 66 bp, Artificial
  Glnpr1191_Primer
atataccggtctcctcttcctcctcgtcctcctgatcctcctgacctgagacagggaggagggaag

SEQ ID NO: 25 - DNA, 66 bp, Artificial
  Glnpr1192_Primer
atataccggtctcctcttcctcctcgtcctcctgatcctcctgacctgagccagggaggagggaag

SEQ ID NO: 26 - DNA, 74 bp, Artificial
  Glnpr1193_Primer
atataccggtctcctcttcctcctcgtcctcctgatcctcctgacctgagccaggtagcagggaagggaagaag

SEQ ID NO: 27 - DNA, 77 bp, Artificial
  Glnpr1237_Primer
ggcggctagcgtttaaacttaagcttccttggaggacccagtacccggatctagagtagttactgcacctttctttg

SEQ ID NO: 28 - DNA, 40 bp, Artificial
  Glnpr1238_Primer
atcggatatcgcctggatcctgtgcaataaggacagggtc

SEQ ID NO: 29 - DNA, 35 bp, Artificial
  Glnpr1239_Primer
gtggcgatatcgcctggatccthtgcaataaggac

SEQ ID NO: 30 - DNA, 57 bp, Artificial
  Glnpr1240_Primer
tggcgatatcgcctggatcctgtgcaataaggacagccttagccaggtgccttaaag

SEQ ID NO: 31 - DNA, 57 bp, Artificial
  Glnpr1241_Primer
tggcgatatcgcctggatcctgtgcaataaggacagggttctccaggtgccttaaag

SEQ ID NO: 32 - DNA, 57 bp, Artificial
  Glnpr1242_Primer
tggcgatatcgcctggatcctgtgcaataaggacagggcaagccaggtgccttaaag

SEQ ID NO: 33 - DNA, 57 bp, Artificial
  Glnpr1243_Primer
tggcgatatcgcctggatcctgtgcaataaggacagcgtaggccaggtgccttaaag

SEQ ID NO: 34 - DNA, 60 bp, Artificial
  Glnpr1244_Primer
gcgatatcgcctggatcctgtcccctaaggactcggttagccaggtgccttaaagctgtg

SEQ ID NO: 35 - DNA, 60 bp, Artificial
  Glnpr1245_Primer
gcgatatcgcctggatcctgtgcaatcctcccagggttagccaggtgccttaaagctgtg

SEQ ID NO: 36 - DNA, 60 bp, Artificial
  Glnpr1246_Primer
gcgatatcgcctggatcctgttccctcctccctcggttagccaggtgccttaaagctgtg

SEQ ID NO: 37 - DNA, 39 bp, Artificial
  Glnpr1285_Primer
cggaagaattcagccacagctttaaggcacctggctaac

SEQ ID NO: 38 - DNA, 3362 bp, Artificial
  GSC2250/GSC2246_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 39 - DNA, 3380 bp, Artificial
  GSC2329_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctgtctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 40 - DNA, 3380 bp, Artificial
  GSC2330_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 41 - DNA, 3380 bp, Artificial
  GSC2323_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctgctacctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 42 - DNA, 3227 bp, Artificial
  GSC2619_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 43 - DNA, 2743 bp, Artificial
  GSC2781_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 44 - DNA, 3362 bp, Artificial
  GSC2342_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctgtctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 45 - DNA, 3380 bp, Artificial
  GSC2328_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctgtctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttgccttgcctgctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 46 - DNA, 3380 bp, Artificial
  GSC2321_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctgtctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 47 - DNA, 3380 bp, Artificial
  GSC2324_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctgtctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctgctacctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 48 - DNA, 3362 bp, Artificial
  GSC2339_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttgttgccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 49 - DNA, 3380 bp, Artificial
  GSC2334_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttgttgccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 50 - DNA, 3380 bp, Artificial
  GSC2336_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttgttgccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctgctacctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 51 - DNA, 3362 bp, Artificial
  GSC2340_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 52 - DNA, 3380 bp, Artificial
  GSC2331_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctgtctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 53 - DNA, 3380 bp, Artificial
  GSC2453_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttgccttgcctgctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 54 - DNA, 3380 bp, Artificial
  GSC2325_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 55 - DNA, 3380 bp, Artificial
  GSC2332_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctgctacctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 56 - DNA, 3362 bp, Artificial
  GSC2341_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttgccttgcctgctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 57 - DNA, 3380 bp, Artificial
  GSC2326_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttgccttgcctgctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttgccttgcctgctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 58 - DNA, 3380 bp, Artificial
  GSC2454_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttgccttgcctgctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 59 - DNA, 3380 bp, Artificial
  GSC2327_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttgccttgcctgctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctgctacctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 60 - DNA, 3344 bp, Artificial
  GSC2338_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 61 - DNA, 3362 bp, Artificial
  GSC2335_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctgtctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 62 - DNA, 3362 bp, Artificial
  GSC2333_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttgccttgcctgctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 63 - DNA, 3362 bp, Artificial
  GSC2337_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 64 - DNA, 3362 bp, Artificial
  GSC2322_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctgctacctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 65 - DNA, 3362 bp, Artificial
  GSC2617_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 66 - DNA, 3209 bp, Artificial
  GSC2739_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 67 - DNA, 2725 bp, Artificial
  GSC2782_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 68 - DNA, 3362 bp, Artificial
  GSC2621_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 69 - DNA, 3209 bp, Artificial
  GSC2740_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 70 - DNA, 2725 bp, Artificial
  GSC2783_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 71 - DNA, 3362 bp, Artificial
  GSC2622_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagtccttaggggacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 72 - DNA, 3209 bp, Artificial
  GSC2742_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagtccttaggggacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 73 - DNA, 2725 bp, Artificial
  GSC2784_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagtccttaggggacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 74 - DNA, 3362 bp, Artificial
  GSC2620_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcctacgctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 75 - DNA, 3209 bp, Artificial
  GSC2737_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcctacgctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 76 - DNA, 3362 bp, Artificial
  GSC2615_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggagaaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 77 - DNA, 3209 bp, Artificial
  GSC2743_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcttgccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 78 - DNA, 3209 bp, Artificial
  GSC2738_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggagaaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 79 - DNA, 3362 bp, Artificial
  GSC2618_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaggctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 80 - DNA, 3209 bp, Artificial
  GSC2975_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaggctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 81 - DNA, 3362 bp, Artificial
  GSC2613_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcagaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 82 - DNA, 3497 bp, Artificial
  GSC2614_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagagtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 83 - DNA, 3362 bp, Artificial
  GSC2741_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagagtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 84 - DNA, 2743 bp, Artificial; identical to SEQ ID NO: 43
  GSC2780_Construct

SEQ ID NO: 85 - DNA, 908 bp, Artificial
  I4(5Y)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacaggatccaggcgatatcgccaccatg

SEQ ID NO: 86 - DNA, 908 bp, Artificial
  I4(9Y nude)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgtctccttctgggacaggatccaggcgatatcgccaccatg

SEQ ID NO: 87 - DNA, 908 bp, Artificial
  I4(7Y nude)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgactccttcggggacaggatccaggcgatatcgccaccatg

SEQ ID NO: 88 - DNA, 908 bp, Artificial
  I4(5Y-5)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcacaggatccaggcgatatcgccaccatg

SEQ ID NO: 89 - DNA, 908 bp, Artificial
  I4(5Y nude)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagtccttaggggacaggatccaggcgatatcgccaccatg

SEQ ID NO: 90 - DNA, 908 bp, Artificial
  I4(3Y nude)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagacctgaggggacaggatccaggcgatatcgccaccatg

SEQ ID NO: 91 - DNA, 908 bp, Artificial
  I4(1Y nude)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagagcagaggggacaggatccaggcgatatcgccaccatg

SEQ ID NO: 92 - DNA, 908 bp, Artificial
  I4(0Y)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacaggatccaggcgatatcgccaccatg

SEQ ID NO: 93 - DNA, 908 bp, Artificial
  I4(5Y-b-ct)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggagaaccctgtccttattgcacaggatccaggcgatatcgccaccatg

SEQ ID NO: 94 - DNA, 908 bp, Artificial
  I4(5Y-b-y)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaggctgtccttattgcacaggatccaggcgatatcgccaccatg

SEQ ID NO: 95 - DNA, 908 bp, Artificial
  I4(5Y-b-2)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcctacgctgtccttattgcacaggatccaggcgatatcgccaccatg

SEQ ID NO: 96 - DNA, 908 bp, Artificial
  I4(5Y-b-a)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcttgccctgtccttattgcacaggatccaggcgatatcgccaccatg

SEQ ID NO: 97 - DNA, 908 bp, Artificial
  I4(5Y-A)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcaaaggatccaggcgatatcgccaccatg

SEQ ID NO: 98 - DNA, 908 bp, Artificial
  I4(5Y-5,G)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcagaggatccaggcgatatcgccaccatg

SEQ ID NO: 99 - DNA, 908 bp, Artificial
  I4(5Ynude,A)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagtccttaggggaaaggatccaggcgatatcgccaccatg

SEQ ID NO: 100 - DNA, 908 bp, Artificial
  I4(5Ynude,b-2)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcctacggagtccttaggggacaggatccaggcgatatcgccaccatg

SEQ ID NO: 101 - DNA, 908 bp, Artificial; identical to SEQ ID NO: 99
  I4(5Ynude,A)_Construct

SEQ ID NO: 102 - DNA, 908 bp, Artificial; identical to SEQ ID NO: 98
  I4(5Y-5,G)_Construct

SEQ ID NO: 103 - DNA, 612 bp, Artificial
  cTNT-I4_LC-HC_Construct
taacggaagaattcagggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtcaggaggatcaggaggacgaggaggaagaggagaccggtgccaccatg

SEQ ID NO: 104 - DNA, 747 bp, Artificial
  cTNT-I5_LC-HC_Construct
taacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatg

SEQ ID NO: 105 - DNA, 128 bp, Artificial
  I4(sh)_LC-HC_Construct
taacggaagaattcagccacagctttaaggcacctggctaacctctgcgcttcttcccttccctcctccctggctcaggtcaggaggatcaggaggacgaggaggaagaggagaccggtgccaccatg

SEQ ID NO: 106 - DNA, 612 bp, Artificial; identical to SEQ ID NO: 103
  cTNT-I4_HC-LC_Construct

SEQ ID NO: 107 - DNA, 747 bp, Artificial; identical to SEQ ID NO: 104
  cTNT-I5_HC-LC_Construct

SEQ ID NO: 108 - DNA, 128 bp, Artificial; identical to SEQ ID NO: 105
  I4(sh)_HC-LC_Construct

SEQ ID NO: 109 - DNA, 3209 bp, Artificial; identical to SEQ ID NO: 80
  GSC2975_Construct

SEQ ID NO: 110 - DNA, 3360 bp, Artificial
  GSC2223_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatcgccaccatgggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtaacggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctcttcgggtctcctggactcagtgccacggttgtccattctgggggtctgtagggagccagcaggagctgcggccgtcctactgaccctgtccttattgcacaggtcaggaggatcaggaggacgaggaggaagaggagaccggtgccaccatggagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 111 - DNA, 3209 bp, Artificial; identical to SEQ ID NO: 66
  GSC3166_Construct

SEQ ID NO: 112 - DNA, 908 bp, Artificial
  I4(0Y; b-a)_Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcttgccgagggaggagggaacaggatccaggcgatatcgccaccatg

SEQ ID NO: 113 - DNA, 908 bp, Artificial
  I4(0Y; b-ct) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggagaaccgagggaggagggaacaggatccaggcgatatcgccaccatg

SEQ ID NO: 114 - DNA, 908 bp, Artificial
  I4(0Y; b-y) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaagggagggaggagggaacaggatccaggcgatatcgccaccatg

SEQ ID NO: 115 - DNA, 908 bp, Artificial
  I4(0Y, b-2) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcctacggagggaggagggaacaggatccaggcgatatcgccaccatg

SEQ ID NO: 116 - DNA, 908 bp, Artificial
  I4(0Y, A) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaaaaggatccaggcgatatcgccaccatg

SEQ ID NO: 117 - DNA, 908 bp, Artificial
  I4(0Y, T) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaataggatccaggcgatatcgccaccatg

SEQ ID NO: 118 - DNA, 908 bp, Artificial
  I4(0Y, G) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaagaggatccaggcgatatcgccaccatg

SEQ ID NO: 119 - DNA, 908 bp, Artificial
  I4(5Ynude; b-a) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcttgccgagtccttaggggacaggatccaggcgatatcgccaccatg

SEQ ID NO: 120 - DNA, 908 bp, Artificial
  I4(5Ynude; b-ct) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaagggagtccttaggggacaggatccaggcgatatcgccaccatg

SEQ ID NO: 121 - DNA, 908 bp, Artificial; identical to SEQ ID NO: 120
  I4(5Ynude; b-y) _Construct

SEQ ID NO: 122 - DNA, 908 bp, Artificial
  I4(5Ynude, T) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagtccttaggggataggatccaggcgatatcgccaccatg

SEQ ID NO: 123 - DNA, 908 bp, Artificial
  I4(5Y-5, b-a) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcttgccctgggaggattgcacaggatccaggcgatatcgccaccatg

SEQ ID NO: 124 - DNA, 908 bp, Artificial
  I4(5Y-5, b-ct) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggagaaccctgggaggattgcacaggatccaggcgatatcgccaccatg

SEQ ID NO: 125 - DNA, 908 bp, Artificial
  I4(5Y-5;b-y) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaggctgggaggattgcacaggatccaggcgatatcgccaccatg

SEQ ID NO: 126 - DNA, 908 bp, Artificial
  I4(5Y-5, b-2) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggcctacgctgggaggattgcacaggatccaggcgatatcgccaccatg

SEQ ID NO: 127 - DNA, 908 bp, Artificial
  I4(5Y-5, A) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcaaaggatccaggcgatatcgccaccatg

SEQ ID NO: 128 - DNA, 908 bp, Artificial
  I4(5Y-5, T) _Construct
ggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactaagcttgaggtgtggcaggcttgagatctggccatacacttgagtgacaatgacatccactttgcctttctctccacaggtgtccactcccacgtccaactgcagctcggttcgatcgataattaattaagctagcgtttaaacttaagcttccttggaggacccagtacccggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcataggatccaggcgatatcgccaccatg

SEQ ID NO: 129 - DNA, 41 bp, Artificial
  I4_Flanking intron
ctaacctctgcgcttcttcccttccctcctccctggctcag

SEQ ID NO: 130 - DNA, 41 bp, Artificial
  I4(22Y+1) _Flanking intron
ctaacctctgcgcttcttcccttccctcctccctgtctcag

SEQ ID NO: 131 - DNA, 41 bp, Artificial
  I4(15Y-5′)_Flanking intron
ctaacctctgcgcttgttgccttccctcctccctggctcag

SEQ ID NO: 132 - DNA, 41 bp, Artificial
  I4(15Y-3′)_Flanking intron
ctaacctctgcgcttcttcccttccctgctacctggctcag

SEQ ID NO: 133 - DNA, 41 bp, Artificial
  I4(22Y-3) _Flanking intron
ctaacctctgcgcttcttgccttgcctgctccctggctcag

SEQ ID NO: 134 - DNA, 23 bp, Artificial
  I4(5Y)_Flanking intron
ctaaccctgtccttattgcacag

SEQ ID NO: 135 - DNA, 23 bp, Artificial
  I4(5Y-5) _Flanking intron
ctaaccctgggaggattgcacag

SEQ ID NO: 136 - DNA, 23 bp, Artificial
  I4(0Y) _Flanking intron
ctaaccgagggaggagggaacag

SEQ ID NO: 137 - DNA, 23 bp, Artificial
  I4(5Ynude) _Flanking intron
ctaaccgagtccttaggggacag

SEQ ID NO: 138 - DNA, 23 bp, Artificial
  I4(5Y-b-2) _Flanking intron
cctacgctgtccttattgcacag

SEQ ID NO: 139 - DNA, 23 bp, Artificial
  I4(5Y-b-a) _Flanking intron
cttgccctgtccttattgcacag

SEQ ID NO: 140 - DNA, 23 bp, Artificial
  I4(5Y-b-ct) _Flanking intron
agaaccctgtccttattgcacag

SEQ ID NO: 141 - DNA, 23 bp, Artificial
  I4(5Y-b-y) _Flanking intron
ctaaggctgtccttattgcacag

SEQ ID NO: 142 - DNA, 23 bp, Artificial
  I4(5Y-G) _Flanking intron
ctaaccctgtccttattgcagag

SEQ ID NO: 143 - DNA, 23 bp, Artificial
  I4(5Y-A) _Flanking intron
ctaaccctgtccttattgcaaag

SEQ ID NO: 144 - DNA, 23 bp, Artificial
  I4(5Y-5-G) _Flanking intron
ctaaccctgggaggattgcagag

SEQ ID NO: 145 - DNA, 23 bp, Artificial
  I4(5Ynude-A) _Flanking intron
ctaaccgagtccttaggggaaag

SEQ ID NO: 146 - DNA, 23 bp, Artificial
  I4(5Ynude-b-2) _Flanking intron
cctacggagtccttaggggacag

SEQ ID NO: 147 - DNA, 23 bp, Artificial
  I4(9Ynude) _Flanking intron
ctaaccgtctccttctgggacag

SEQ ID NO: 148 - DNA, 23 bp, Artificial
  I4(7Ynude) _Flanking intron
ctaaccgactccttcggggacag

SEQ ID NO: 149 - DNA, 23 bp, Artificial
  I4(5Ynude-b-a) _Flanking intron
cttgccgagtccttaggggacag

SEQ ID NO: 150 - DNA, 23 bp, Artificial
  I4(3Ynude) _Flanking intron
ctaaccgagacctgaggggacag

SEQ ID NO: 151 - DNA, 23 bp, Artificial
  I4(1Ynude) _Flanking intron
ctaaccgagagcagaggggacag

SEQ ID NO: 152 - DNA, 23 bp, Artificial
  I4(5Y-T) _Flanking intron
ctaaccctgtccttattgcatag

SEQ ID NO: 153 - DNA, 41 bp, Artificial; identical to SEQ ID NO: 129
  I4sh_Flanking intron

SEQ ID NO: 154 - DNA, 24 bp, Artificial
  I5_Flanking intron
actgaccctgtccttattgcacag

SEQ ID NO: 155 - DNA, 42 bp, Artificial
  I5(22Y) _Flanking intron
actgacctctgcgcttcttcccttccctcctccctggctcag

SEQ ID NO: 156 - DNA, 42 bp, Artificial
  I5(22Y+1) _Flanking intron
actgacctctgcgcttcttcccttccctcctccctgtctcag

SEQ ID NO: 157 - DNA, 42 bp, Artificial
  I5(22Y-3) _Flanking intron
actgacctctgcgcttcttgccttgcctgctccctggctcag

SEQ ID NO: 158 - DNA, 42 bp, Artificial
  I5(15Y-3′) _Flanking intron
actgacctctgcgcttcttcccttccctgctacctggctcag

SEQ ID NO: 159 - DNA, 42 bp, Artificial
  I5(15T-5′) _Flanking intron
actgacctctgcgcttgttgccttccctcctccctggctcag

SEQ ID NO: 160 - DNA, 23 bp, Artificial
  I4(0Y; b-a) _Flanking intron
cttgccgagggaggagggaacag

SEQ ID NO: 161 - DNA, 23 bp, Artificial
  I4(0Y; b-ct) _Flanking intron
agaaccgagggaggagggaacag

SEQ ID NO: 162 - DNA, 23 bp, Artificial
  I4(0Y; b-y) _Flanking intron
ctaagggagggaggagggaacag

SEQ ID NO: 163 - DNA, 23 bp, Artificial
  I4(0Y, b-2) _Flanking intron
cctacggagggaggagggaacag

SEQ ID NO: 164 - DNA, 23 bp, Artificial
  I4(0Y, A) _Flanking intron
ctaaccgagggaggagggaacag

SEQ ID NO: 165 - DNA, 23 bp, Artificial
  I4(0Y, T) _Flanking intron
ctaaccgagggaggagggaacag

SEQ ID NO: 166 - DNA, 23 bp, Artificial
  I4(0Y, G) _Flanking intron
ctaaccgagggaggagggaacag

SEQ ID NO: 167 - DNA, 23 bp, Artificial
  I4(5Ynude; b-ct) _Flanking intron
ctaagggagtccttaggggacag

SEQ ID NO: 168 - DNA, 23 bp, Artificial
  I4(5Ynude; b-y) _Flanking intron
ctaagggagtccttaggggacag

SEQ ID NO: 169 - DNA, 23 bp, Artificial
  I4(5Ynude, T) _Flanking intron
ctaaccgagtccttaggggacag

SEQ ID NO: 170 - DNA, 23 bp, Artificial
  I4(5Y-5, b-a) _Flanking intron
cttgccctgggaggattgcacag

SEQ ID NO: 171 - DNA, 23 bp, Artificial
  I4(5Y-5, b-ct) _Flanking intron
agaaccctgggaggattgcacag

SEQ ID NO: 172 - DNA, 23 bp, Artificial
  I4(5Y-5;b-y) _Flanking intron
ctaaggctgggaggattgcacag

SEQ ID NO: 173 - DNA, 23 bp, Artificial
  I4(5Y-5, b-2) _Flanking intron
cctacgctgggaggattgcacag

SEQ ID NO: 174 - DNA, 23 bp, Artificial
  I4(5Y-5, A) _Flanking intron
ctaaccctgggaggattgcacag

SEQ ID NO: 175 - DNA, 23 bp, Artificial
  I4(5Y-5, T) _Flanking intron
ctaaccctgggaggattgcacag